(19) United States
(12) Patent Application Publication
(10) Pub. No.: US 2026/0218311 A1
(43) Pub. Date: Jul. 30, 2026

(54) Method of Predicting Breast Cancer Recurrence

(71) Applicant: University of Cincinnati, Cincinnati, OH (US)

(72) Inventors: Susan E. Waltz, Blue Ash, OH (US); James Davis, Cincinnati, OH (US); Susanne Wells, Cincinnati, OH (US); Sara Vicente-Munoz, Montgomery, OH (US)

(73) Assignee: University of Cincinnati, Cincinnati, OH (US)

(21) Appl. No.: 19/287,668

(22) Filed: Jul. 31, 2025

Related U.S. Application Data
(60) Provisional application No. 63/677,795, filed on Jul. 31, 2024.

(51) Int. Cl. C12Q 1/6886

(52) U.S. Cl. CPC C12Q 1/6886; C12Q 2600/118; C12Q 2600/158

(57) ABSTRACT

A method for predicting tumor recurrence in a subject diagnosed with cancer is provided. The method involves analyzing a tumor sample obtained from the subject by using an assay to determine the extent of expression of one or more genes selected from a first group in the tumor sample (“Group 1 genes”) and a second group in the tumor sample (“Group 2 genes”). An increased likelihood that the subject will exhibit tumor recurrence is predicted when one or more of the Group 1 genes are upregulated, one or more of the Group 2 genes are downregulated or a combination of Group 1 genes are upregulated and Group 2 genes are downregulated.

### CROSS-REFERENCE TO RELATED APPLICATIONS

This application claims priority to, and the benefit of the filing date of, U.S. Provisional Application No. 63/677,795 filed Jul. 31, 2024, the disclosure of which is incorporated by reference herein in its entirety.

### STATEMENT REGARDING FEDERALLY FUNDED RESEARCH OR DEVELOPMENT

This invention was made with government support under CA239697 awarded by the National Institutes of Health. The government has certain rights in the invention.

### TECHNICAL FIELD

The present invention relates to stratifying breast cancer patients that may benefit from additional treatment.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art. Advances in treatments have resulted in an overall 90% 5-year survival rate for breast cancer patients. However, a large gap remains between survival rates of those with localized versus distant metastatic breast cancer. While localized breast cancer has a 98% survival rate, distant metastatic breast cancer has a 31% survival rate. In addition, the ten-year survival rate for breast cancer decreases from 85% to 46.9% for local recurrence and to less than 5% for distant recurrence. This highlights an unmet need to understand markers and drivers of metastatic and recurrent breast cancer to improve currently unacceptable outcomes.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention.

In an embodiment of the invention, a method for predicting tumor recurrence in a subject diagnosed with cancer is provided. The method involves analyzing a tumor sample obtained from the subject by using an assay to determine the extent of expression of one or more genes selected from the group consisting of ACOT7, ACOT13, ME1, ANGPTL4, DHCR7, SRD5A1, HSD17B2, UGT8, LCLAT1, PLAAT1, DGAT2, ARSK and NEU4 in the tumor sample (“Group 1 genes”). In addition, the method analyzes the tumor sample by using an assay to determine the extent of expression of one or more genes selected from the group consisting of OLAH, ACACB, IVD, FASN, ACSL6, ELOVL5, CPT1C, CPT1B, ACADSB, ACAA2, ACOX1, PTGR1, FAAH, ACSF, BDH2, ACOT11, MBOAT1, MVK, STARD3, OSBPL5, NCOA1, AGPAT3, AGPAT5, GPAT4, INPP4B, ETNK1, PLAAT5, PITPNM2, SLC44A4, APOA5, GPAT2, MGLL and PLD6 in the tumor sample (“Group 2 genes”). An increased likelihood that the subject will exhibit tumor recurrence is predicted when one or more of the Group 1 genes are upregulated, one or more of the Group 2 genes are downregulated or a combination of Group 1 genes are upregulated and Group 2 genes are downregulated.

In one embodiment, a combination of at least two genes selected from the Group 1 genes, the Group 2 genes, or both Group 1 genes and Group 2 genes are utilized for predicting an increased likelihood of tumor recurrence. In another embodiment, a combination of at least four genes selected from the Group 1 genes, the Group 2 genes, or both Group 1 genes and Group 2 genes are utilized for predicting an increased likelihood of tumor recurrence. In one embodiment, the four genes are DHCR7, BDH2, ELOVL5, and ARSK.

In another embodiment, a combination of at least eight genes selected from the Group 1 genes, the Group 2 genes, or both Group 1 genes and Group 2 genes are utilized for predicting an increased likelihood of tumor recurrence. In one embodiment, a combination of at least ten genes selected from the Group 1 genes, the Group 2 genes, or both Group 1 genes and Group 2 genes are utilized for predicting an increased likelihood of tumor recurrence. In another embodiment, a combination of at least fourteen genes selected from the Group 1 genes, the Group 2 genes, or both Group 1 genes and Group 2 genes are utilized for predicting an increased likelihood of tumor recurrence.

In another embodiment, a combination of the genes are utilized for predicting tumor recurrence prior to or after cancer treatments. In one embodiment, the cancer is selected from the group consisting of lung cancer, prostate cancer, testicular cancer, brain cancer, skin cancer, colon cancer, rectal cancer, gastric cancer, esophageal cancer, tracheal cancer, head and neck cancer, pancreatic cancer, liver cancer, breast cancer, ovarian cancer, lymphoid cancer, kidney cancer, cervical cancer, bone cancer, vulvar cancer and melanoma. In another embodiment, the cancer is breast cancer. In one embodiment, the cancer is node positive or node negative breast cancer.

In another embodiment, a combination of gene sets identified in Table 1 are used as a diagnostic for metastasis, tumor recurrence, or both, prior to or after cancer treatment. In one embodiment, the tumor sample is a blood sample. In another embodiment, the method also includes modifying the subject's cancer therapy regimen based on the prediction of an increased likelihood of tumor recurrence.

### BRIEF DESCRIPTION OF THE DRAWINGS

The objects and advantages of the disclosed invention will be further appreciated in light of the following detailed descriptions and drawings in which:

FIG. 1A is a graph showing a full ¹H spectrum acquired in a Bruker Avance spectrometer operating at 600 MHz. 512 transients were acquired at 288 K, apodized using 1-Hz line broadening exponential, Fourier transformed, phased and baseline corrected.

FIG. 1B is a graph showing the expansion of different regions of the ¹H spectrum (0.70-1.60 ppm) of the total lipid fraction showing the assignments of the lipid classes identified. C: cholesterol; FA: fatty acids; PC: phosphocholine; SM: sphingomyelin; BHT: butylated hydroxytoluene (solvent, antioxidant).

FIG. 1C is a graph showing the expansion of different regions of the ¹H spectrum (1.75-2.40 ppm) of the total lipid fraction showing the assignments of the lipid classes identified. C: cholesterol; FA: fatty acids; PC: phosphocholine; SM: sphingomyelin; BHT: butylated hydroxytoluene (solvent, antioxidant).

FIG. 1D is a graph showing the expansion of different regions of the ¹H spectrum (2.70-3.70 ppm) of the total lipid fraction showing the assignments of the lipid classes identified. C: cholesterol; FA: fatty acids; PC: phosphocholine; SM: sphingomyelin; BHT: butylated hydroxytoluene (solvent, antioxidant).

FIG. 1E is a graph showing the expansion of different regions of the ¹H spectrum (3.80-4.60 ppm) of the total lipid fraction showing the assignments of the lipid classes identified. C: cholesterol; FA: fatty acids; PC: phosphocholine; SM: sphingomyelin; BHT: butylated hydroxytoluene (solvent, antioxidant).

FIG. 1F is a graph showing the expansion of different regions of the ¹H spectrum (4.90-6.00 ppm) of the total lipid fraction showing the assignments of the lipid classes identified. C: cholesterol; FA: fatty acids; PC: phosphocholine; SM: sphingomyelin; BHT: butylated hydroxytoluene (solvent, antioxidant).

FIG. 2A is a schematic showing a cholesterol biosynthesis pathway. Multistep reactions are illustrated with discontinuous arrows. Relevant enzymes from the Kmplot.com-derived lipid metabolism gene signature are shown where appropriate, with an upward arrow indicating that gene induction is associated with worse patient outcomes and a downward arrow indicating that gene suppression is associated with worse patient outcomes.

FIG. 2B is a schematic showing the chemical structure of cholesterol. The carbon positions highlighted in red represent the cholesterol atoms quantified by NMR and compared between cell lines.

FIG. 2C is a graph showing a comparison of 1D ¹H-NOESY spectra of the lipid fraction from R7, R7sgRON, R7shDEK corresponding with the cholesterol peaks analyzed by ¹H-NMR.

FIG. 2D is a graph showing the intracellular fold change of cholesterol measured from C26, C27-CH₃ and C18-CH₃ carbon positions and quantified by ¹H-NMR and expressed as a fold change of the control group (R7). Bar plots indicate mean±SEM. Replicates of each cell line are shown as individual dots (n=7). Statistical significance was assessed using one-way ANOVA. *P≤0.05; **P≤0.01; ***P≤0.001; ****P≤0.0001. PM: plasma membrane; MVK: Mevalonate Kinase; DHCR7: 7-Dehydrocholesterol Reductase; STARD3: StAR-Related Lipid Transfer Domain Containing 3; OSBPL5: Oxysterol Binding Protein Like 5.

FIG. 3A is a schematic showing the synthesis of sphingomyelin and other derivatives of ceramide. Translation products from the Kmplot.com-derived lipid metabolism gene signature have been added where appropriate, with an upward arrow indicating that upregulation of this gene is associated with worse patient outcomes.

FIG. 3B is a graph showing intracellular fold change of sphingomyelin measured from sphingomyelin olefinic CH ¹H-NMR. Bar plots indicate mean±SEM. Replicates of each cell line are shown as individual dots (n=7). Statistical significance was assessed using one-way ANOVA. *P<0.05; **P<0.01; ***P<0.001; ****P<0.0001. GM4: N-Acetylneuraminyl-galactosylceramide; NEU4: N-Acetyl-Alpha-Neuraminidase 4; UGT8: UDP Glycosyltransferase 8; SMS: sphingomyelin synthase.

FIG. 4A is a schematic representation of metabolic processes involved in the production of glycerol, glycerophospholipids, and fatty acids. Translation products from the KMplot.com-derived lipid metabolism gene signature have been added where appropriate, with an upward arrow indicating that upregulation of this gene is associated with worse patient outcomes and a downward arrow indicating that downregulation of this gene is associated with better patient outcomes.

FIG. 4B is a graph showing the intracellular fold change of the protons located at positions C1 and C3 of the glycerol backbone (CH₂OR1-CHOR2-CH₂OR3) of the TAG.

FIG. 4C is a graph showing the glyceryl C3H₂ group (CH₂OR1-CHOR2-CH₂—X) of glycerophospholipids.

FIG. 4D is a pair of graphs showing the NMR peaks derived from free glycerol, both C1H3 and C3H3 and C2H2 resonances.

FIG. 4E is a graph showing an analysis of the bis-allylic (—CH₂) groups, which is specific for linoleic acid, the olefinic (—CH) and α-methylenes fatty acids group of the acyl chain.

FIG. 4F is a schematic showing the chemical structure of a representative fatty acid. The blue dots represent the carbon position analyzed by ¹H-NMR. Bar plots indicate mean±SEM. Replicates of each cell line are shown as individual dots (n=7). Statistical significance was assessed using one-way ANOVA. *P≤0.05; **P≤0.01; ***P≤0.001; ****P≤0.0001. MGLL: monoglyceride lipase; DGAT2: Diacylglycerol O-Acyltransferase 2; GPAT2: 1-Acylglycerol-3-Phosphate O-Acyltransferase 2; GPAT4: glycerol-3-phosphate acyltransferase 4; AGPAT3: 1-acylglycerol-3-phosphate O-acyltransferase 3; AGPAT5: 1-Acylglycerol-3-Phosphate O-Acyltransferase 5; MBOAT1: Membrane Bound O-Acyltransferase Domain Containing 1.

FIG. 5A is a graph showing the results of a query of Gene Expression Omnibus-derived KMplot breast cancer datasets regarding Overall survival (OS).

FIG. 5B is a graph showing the results of a query of Gene Expression Omnibus-derived KMplot breast cancer datasets regarding Distant Metastasis Free Survival (DMFS).

FIG. 5C is a graph showing the results of a query of Gene Expression Omnibus-derived KMplot breast cancer datasets regarding Post-Progression Free Survival (PPS).

FIG. 5D is a graph showing the results of a query of Gene Expression Omnibus-derived KMplot breast cancer datasets regarding the Recurrence Free Survival (RFS) of breast cancer patients stratified by expression of the lipid gene signature (Table 1).

FIG. 6A is a graph showing the gene signature developed on the KMplot cancer datasets (Table 1) tested against a breast cancer dataset from the Cancer Genome Atlas (TCGA) Pan Cancer datasets to examine overall survival (OS).

FIG. 6B is a graph showing the gene signature developed on the KMplot cancer datasets (Table 1) tested against a breast cancer dataset from the Cancer Genome Atlas (TCGA) Pan Cancer datasets to examine Progression Free Survival (PFS).

FIG. 6C is a graph showing the gene signature developed on the KMplot cancer datasets (Table 1) tested against a Gene Expression Omnibus-derived KMplot ovarian cancer dataset to examine overall survival (OS).

FIG. 6D is a graph showing the gene signature developed on the KMplot cancer datasets (Table 1) tested against a Gene Expression Omnibus-derived KMplot lung cancer dataset to examine overall survival (OS).

FIG. 7A is a graph showing a query of Gene Expression Omnibus-derived KMplot breast cancer datasets for Overall survival (OS) of breast cancer patients stratified by expression of the lipid gene signature (Table 1).

FIG. 7B is a graph showing a query of Gene Expression Omnibus-derived KMplot breast cancer datasets for Recurrence Free Survival (RFS) of breast cancer patients stratified by expression of the lipid gene signature (Table 1).

FIG. 7C is a graph showing a query of Gene Expression Omnibus-derived KMplot breast cancer datasets for Distant Metastasis Free Survival (DMFS) of breast cancer patients stratified by expression of the lipid gene signature (Table 1).

FIG. 7D is a graph showing a query of Gene Expression Omnibus-derived KMplot breast cancer datasets for Overall survival (OS) of breast cancer patients stratified by expression of the lipid gene signature (Table 1).

FIG. 7E is a graph showing a query of Gene Expression Omnibus-derived KMplot breast cancer datasets for Recurrence Free Survival (RFS) of breast cancer patients stratified by expression of the lipid gene signature (Table 1).

FIG. 7F is a graph showing a query of Gene Expression Omnibus-derived KMplot breast cancer datasets for Distant Metastasis Free Survival (DMFS) of breast cancer patients stratified by expression of the lipid gene signature (Table 1).

FIG. 8A is a graph showing receiver-operator characteristic (ROC) analysis of breast cancer patient complete response to chemotherapy stratified by expression of genes in the lipid gene signature (Table 1) found to be upregulated in recurrent breast cancer.

FIG. 8B is a graph showing Receiver-operator characteristic (ROC) analysis of breast cancer patient recurrence free survival (RFS) to chemotherapy stratified by expression of genes in the gene signature found to be downregulated in recurrent breast cancer.

FIG. 8C is a graph showing Receiver-operator characteristic (ROC) analysis of node-positive breast cancer patient RFS to therapy stratified by expression of genes in the gene signature found to be upregulated in recurrent breast cancer.

FIG. 8D is a graph showing Receiver-operator characteristic (ROC) analysis of node-positive breast cancer patient RFS to therapy stratified by expression of genes in the gene signature found to be downregulated in recurrent breast cancer.

FIG. 9A is a graph showing a refined gene expression signature for lipid metabolisms based on KMplot.com GEO-derived breast cancer dataset. Gene Expression Omnibus-derived KMplot breast cancer datasets were used to query Overall survival (OS).

FIG. 9B is a graph showing a refined gene expression signature for lipid metabolisms based on KMplot.com GEO-derived breast cancer dataset. Gene Expression Omnibus-derived KMplot breast cancer datasets were used to query Distant Metastasis Free Survival (DMFS).

FIG. 9C is a graph showing a refined gene expression signature for lipid metabolisms based on KMplot.com GEO-derived breast cancer dataset. Gene Expression Omnibus-derived KMplot breast cancer datasets were used to query Post-Progression Survival (PPS).

FIG. 9D is a graph showing a refined gene expression signature for lipid metabolisms based on KMplot.com GEO-derived breast cancer dataset. Gene Expression Omnibus-derived KMplot breast cancer datasets were used to query Recurrence Free Survival (RFS) of breast cancer patients stratified by expression of the four gene lipid gene signature (Table 2).

FIG. 10A is a graph showing Multivariate statistical analysis by principal component analysis (PCA) distinguishes cell lines based on their lipid profile. (A) PCA scatter plot shows distinct clustering of the three cell lines (R7, R7sgRON and R7shDEK) according to their lipid composition, indicating a clear difference in their lipidomes.

FIG. 10B is a graph showing a PCA scatterplot for R7 (n=7).

FIG. 10C is a graph showing a PCA scatterplot for R7sgRON (n=7).

FIG. 10D is a graph showing a PCA scatterplot for R7shDEK (n=7). Prior to PCA, the data were scaled to unite variance to ensure equal contribution from each variable. This distinct clustering pattern suggests that the cell lines exhibit unique lipid signature.

FIG. 11 is a graph showing breast cancer patient response to therapy compared to the four gene expression signature from ROCplot.com. Receiver-operator characteristic (ROC) analysis of breast cancer patient complete response to chemotherapy stratified by expression of genes in the four gene signature (Table 2).

### DEFINITIONS

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “cancer treatment” means any cancer treatment known in the art including, but not limited to, surgery, chemotherapy and radiation therapy.

As used herein, the term “downregulation” means detecting a decrease in the amount or activity of a gene or gene product relative to a baseline or control state, through any mechanism including, but not limited to decreased transcription, translation and/or decreased stability of the transcript or protein product

As used herein, the term “upregulation” means detecting an increase in the amount or activity of a gene or gene product relative to a baseline or control state, through any mechanism including, but not limited to increased transcription, translation and/or increased stability of the transcript or protein product.

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In one embodiment, the present invention involves two genes that have already been associated with poor outcomes in patients with breast cancer. RON is a receptor tyrosine kinase that is overexpressed in more than 50% of breast cancers independent of subtype, and stimulates cancer stemness, metastasis and recurrence. DEK is a chromatin-associated oncogene that also promotes breast cancer stemness and metastasis and can be stimulated by RON signaling. Together, high DEK and RON expression levels were strongly linked to poor outcomes in human subjects. Previous studies have shown that fatty acids can regulate tumor cell growth, invasion, and progression both positively and negatively. In addition, increases in cholesterol biosynthesis and cholesterol levels have been associated with RON-driven Breast Cancer Stem Cell (BCSC) phenotypes. Lipid metabolism is the new frontier of cancer biomarkers and targets and might be a potential driver of RON and DEK associated metastasis and recurrence.

To determine whether RON and DEK upregulation in breast cancer is required for lipid metabolism that is linked to poor outcomes, ¹H-NMR (Nuclear Magnetic Resonance) spectroscopy was used to determine relative abundances of different intact lipids in families defined by (i) headgroup (phosphatidylcholine (PC), phosphatidylethanolamine (PE)) and average degree of fatty acyl chain unsaturation, (ii) sphingomyelin and (iii) cholesterol, rather than individual lipid species. This provides a broad overview of the complex lipids detectable in cells. It was found that breast cancer cells with RON and DEK loss of function harbor independent dysregulation of defined lipid metabolites, including cholesterol, unsaturated fatty acids, glycerol, sphingomyelin, and glycerophospholipids. By examining relevant transcriptionally regulated enzymes, a lipid metabolism related gene signature was identified that predicts breast cancer recurrence, and also poor outcomes for lung, ovarian, and gastric cancer. These data also support a role for RON and DEK in energy metabolism to support breast cancer recurrence and metastasis.

RON and DEK are implicated in breast cancer metastasis and recurrence, and since these same phenotypes have been linked to lipid dysregulation, the gene signature discovered here might have diagnostic and prognostic potential for patients with breast and other cancer types. As described herein, 1H-NMR spectroscopy was employed as an exploratory tool to identify dysregulated lipid families and characterize the lipid metabolic profile of murine breast cancer cells with respect to loss of RON or DEK expression. Specifically, global information on glycerophospholipid head groups and backbones, acyl chain length, and degree of unsaturation of fatty acids was obtained, as well as cholesterol and sphingolipid content. Although NMR-based methods are generally less sensitive than MS and can be limited by overlapping signals, the present invention used a simple and efficient NMR-based lipidomic method to provide a non-destructive comprehensive lipid profile in a single experiment with minimal sample processing and without multiple internal standards.

A wealth of evidence has shown that lipid metabolism is important in metastasis and recurrence of breast cancer. For instance, cholesterol biosynthesis inhibition using statins lowered the risk of recurrence. The present inventors have found that RON signaling increases cholesterol levels, and that RON-stimulated metastasis and recurrence can be suppressed by statins. In addition, fatty acid synthesis promotes DNA damage and ROS production in residual breast cancer cells. Although the exact roles that DNA damage and ROS play in cancer are unclear, the data presented in the gene signature herein implies that fatty acid synthesis suppresses cancer progression, perhaps due to modulation of DNA damage and ROS levels. Independent of RON, DEK appears to lower total fatty acid levels, as a possible contributor to the pro-tumor capabilities of DEK in breast cancer. Although clinical data is lacking, trends in the published cellular literature indicate that sphingolipids and phospholipids also play key roles in breast cancer recurrence and metastasis. RON, independent of DEK, seems to increase sphingomyelin levels. The literature combined with the lipid data presented herein supports roles for RON and DEK in the reprogramming of breast cancer cells towards a cancer stem cell phenotype. Specifically, RON appears to promote cholesterol and sphingomyelin levels, while DEK suppresses cholesterol and total fatty acid levels.

Polar fractions from the above cell lines have been studied. Integrating the polar and nonpolar data increases our understanding of the impact of RON and DEK on pathways that feed into lipid metabolism. For instance, the inventors previously found that pyruvate levels were suppressed by RON but stimulated by DEK. Pyruvate can be metabolized into glycerol, which also shows a similar pattern as pyruvate with suppression by RON, and stimulation by DEK in the absence of glycerol changes. This indicates that RON and DEK may independently regulate the flux of pyruvate into different pathways. In addition, pyruvate can be converted into acetyl-CoA and used for fatty acid and cholesterol synthesis. Other contributors to acetyl-CoA levels that were measured include isoleucine, which was found to be increased by both DEK and RON; glutamate, which was not affected by RON or DEK knock-down; citrate, which was found to be suppressed by both RON and DEK; and acetate, which was found to be increased by both RON and DEK. However, in the absence of tracer experiments, it is difficult to determine the relative contributions of these different pathways to lipid synthesis.

For phospholipids, the present inventors found that phosphatidylcholine levels were stimulated by RON and DEK, while glycerophosphatidylcholine was suppressed by RON and stimulated by DEK. The observed dysregulation of specific phospholipids in the absence of RON occurred without any change in total phospholipid levels, and the lack of a change in the glycerol signature. In addition, we observed alterations in sphingolipid levels, the degree of unsaturation, and cholesterol, which is importantly involved in membrane lipid raft function. This suggests that RON and DEK may control pathways that influence the physical properties of membranes including fluidity and signaling, important possible contributors to metastasis and recurrence in breast cancer.

There is a need for additional studies of the functional roles of genes as potential drivers of breast cancer metastasis and recurrence. For example, the hazard ratio for the ACADSB RFS curve alone was 0.54, which indicates that patients with high ACADSB levels in their tumors are half as likely to have recurrence compared to patients with low ACADSB, thus supporting a potential high value of this gene as a predictive biomarker and driver of mortality. To define the significance of the gene signature, node +ve breast cancers were focused on, which have already spread to lymph nodes and carry a high likelihood of recurrence. Focusing on the group of 47 genes (Table 1) that were indicative of improved response to treatment in node +ve patients, patients at a higher risk of recurrence and non-response to treatment were identified. In one embodiment of the present invention, this gene signature was narrowed down to a combination of only four genes which fully retain the predictive power of the previous signature. At least some of these genes appear to be functionally involved in cancer phenotypes that portend poor outcome, and in this case, their therapeutic modulation may result in new avenues to suppress breast cancer recurrence and/or treatment resistance. Lipid profiles identified in sgRON and shDEK murine cell lines are the foundation for future research in animal models of breast cancer recurrence and metastasis. Data validation in human models is now critical to advance lipid related biomarkers and targets towards clinical application.

### Lipidomic Profile of Breast Cancer by 1H-NMR Spectroscopy

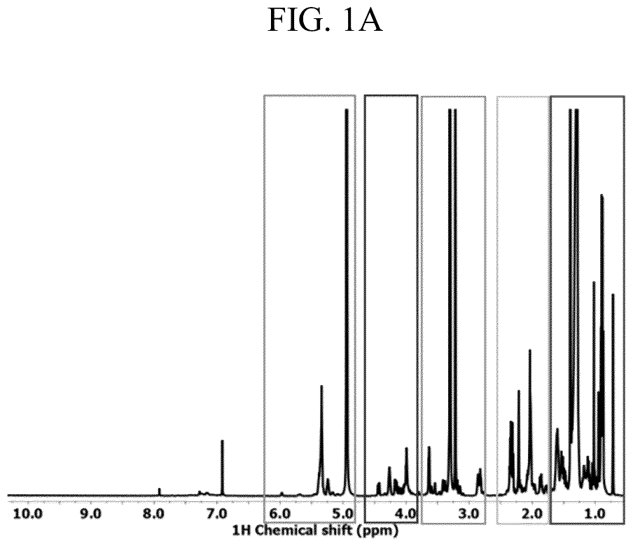

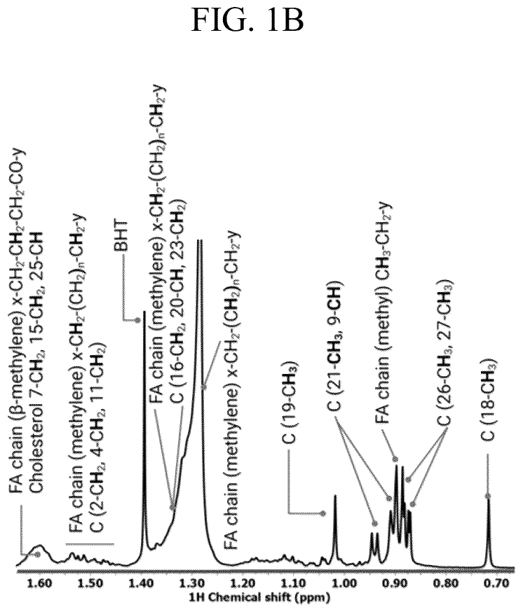

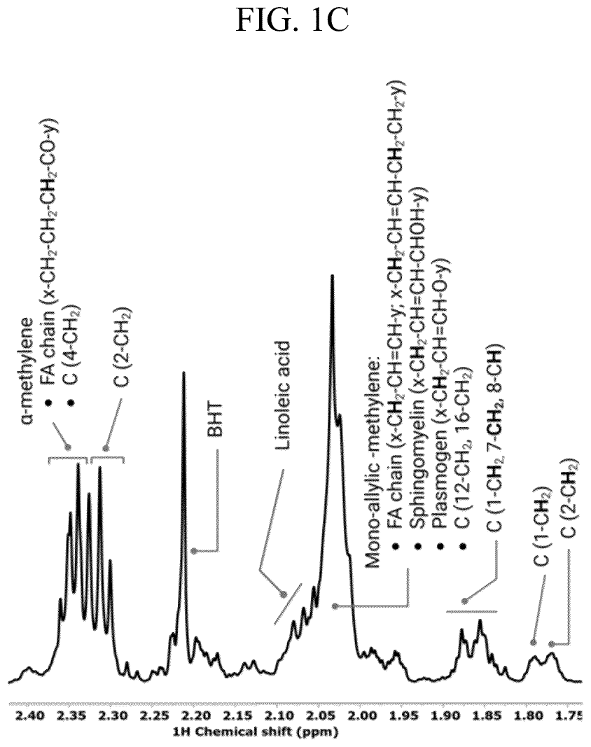

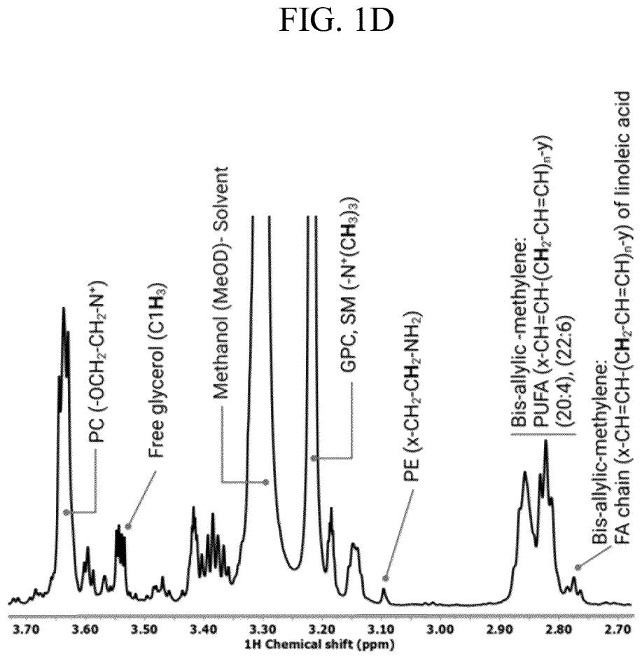

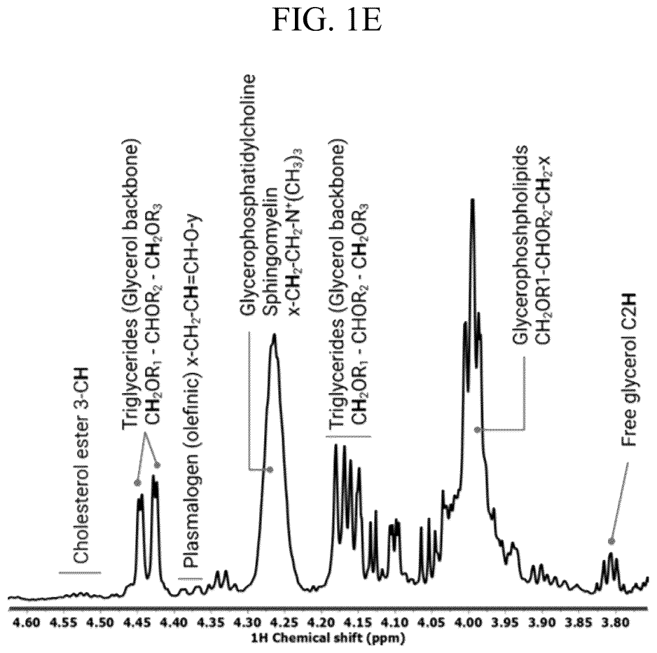

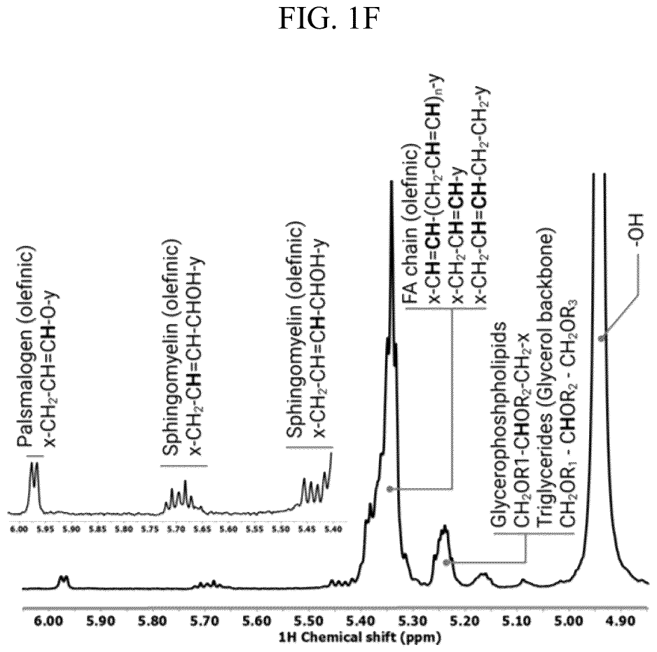

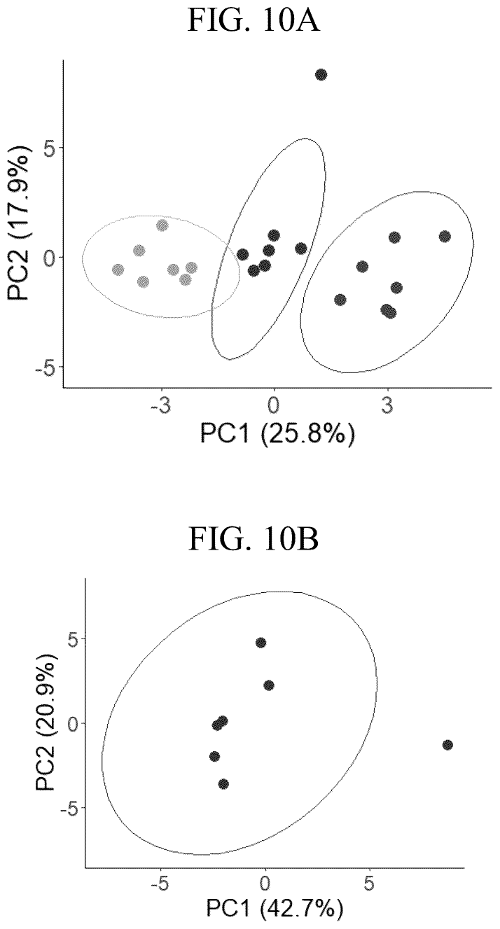

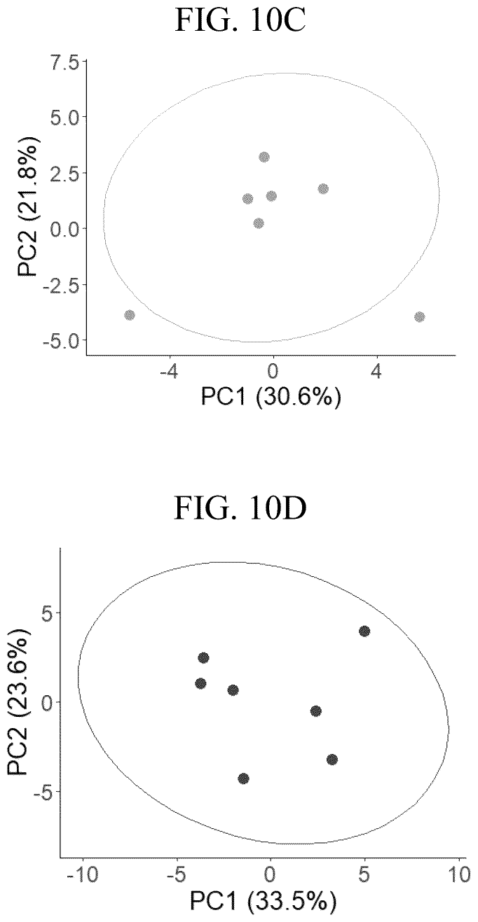

To qualitatively and quantitatively define RON- and DEK-dependent reprogramming of lipid metabolites, NMR spectroscopy was applied to R7 breast cancer cells and the corresponding RON or DEK knockdown cell lines. Specifically, proton (1H) NMR determined lipid composition in the presence and absence of RON and DEK. Despite the inherent width of individual lipid resonances, as well as the wide variety and complexity of the lipid species, good-quality spectra were obtained for all samples. A representative 1D 1H NMR spectrum of the total lipid fraction of R7sgRON is shown in FIG. 1A where the most representative lipid families were identified and assigned. As previously described, the 1H-NMR resonances of the subunits of the different lipid classes were distributed as follows: the methyl (CH3) and methylene (CH2) resonances from cholesterol and the acyl chains are located from 0.65 to 3.00 ppm (FIG. 1B-D). The spectral region between 3.05 and 5.25 ppm (FIG. 1D-F) comprises the phospholipid head group and glycerol backbone moieties of the glycerophospholipids. The vinyl proton peaks resound between 5.30 and 6.00 ppm (FIG. 1F). Therein we determined the relative abundance of different subunits of complex lipids, including sterols (e.g., cholesterol), sphingolipids, glycerophospholipids, and fatty acids. We applied Principal Component Analysis (PCA) as an exploratory tool to assess the separation of the three groups based on their lipid profiles (FIGS. 10A-10D). Clear separation observed in the PCA plot provides visual evidence of distinct lipid patterns among the groups (FIG. 10A).

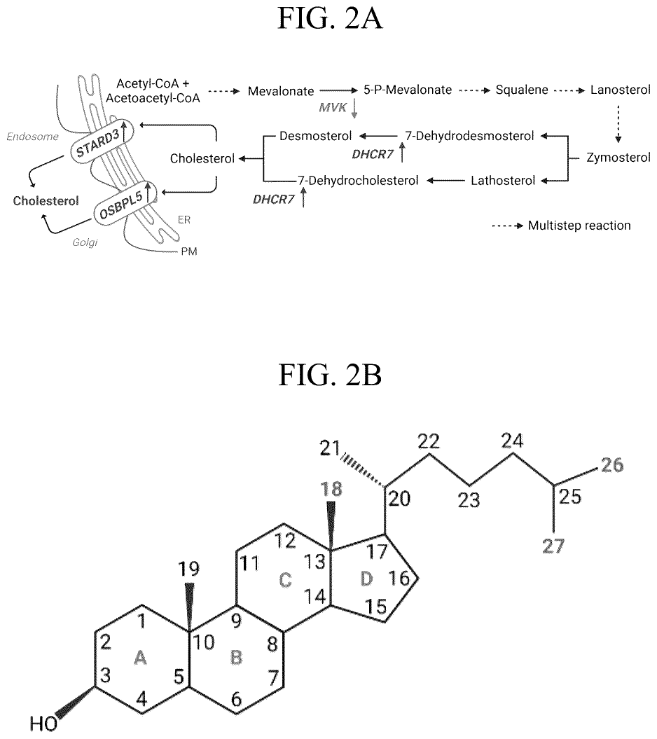

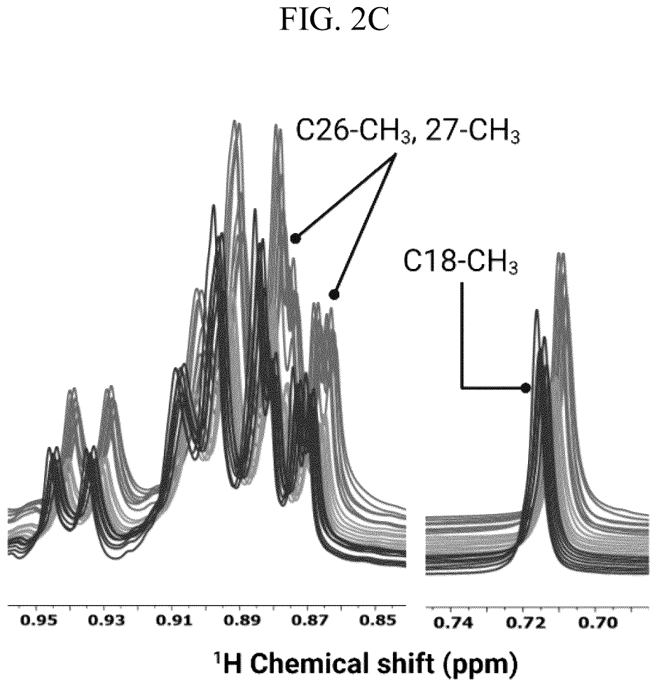

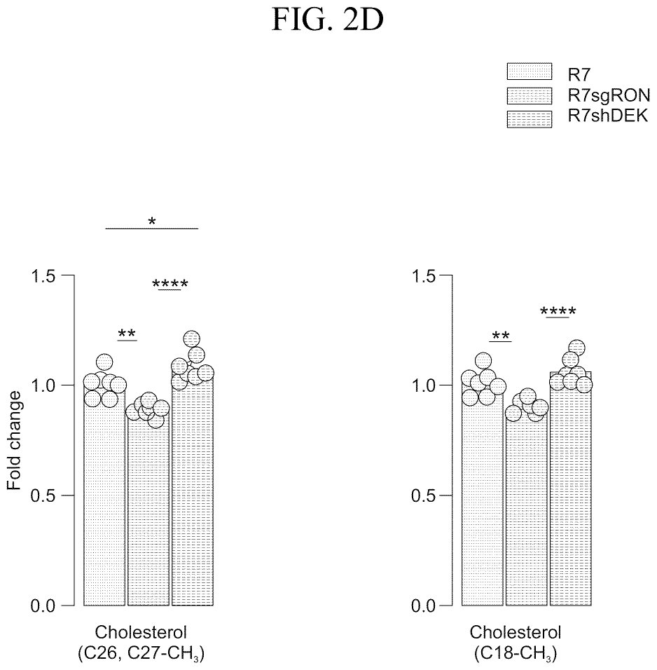

Cholesterol biosynthesis is associated with increased breast cancer stem cell potential and increased recurrence, and entails a series of highly complex reactions that occur in the endoplasmic reticulum (ER). The process begins with Acetyl-CoA and eventually results in a four-ring structure with a side-chain and total of 27-carbons (FIG. 2A, B). Once formed, cholesterol is transported via non-vesicular and vesicular mechanisms. Vesicular transport of cholesterol occurs through organelle membranes, such as in endosomes and the Golgi apparatus, to the cellular membrane with the participation of sterol and oxysterol trafficking proteins (FIG. 2A). Cholesterol upregulation is a poor prognostic factor in breast cancer and is associated with shortened relapse-free survival. Cholesterol also acts as an estrogen receptor agonist via its metabolite 27-hydroxycholesterol, supporting recurrence and metastasis progression. Cholesterol resonances from 1H spectra were integrated, quantified, and used to determine cholesterol levels in the R7, R7sgRON, and R7shDEK cell lines. The methyl (CH3) cholesterol resonance at positions C18, and C26, C27, showed that RON loss decreased cholesterol levels. In contrast, DEK loss did not impact cholesterol levels, and even elevated them compared to the R7 control. (FIG. 2C, D). In line with our previous work wherein RON promoted key genes and enzymes in the cholesterol biosynthesis pathway as well as the incorporation of glucose-derived carbon into cholesterol, this establishes that RON (but not DEK) overexpression contribute to the promotion of cholesterol biosynthesis.

### RON Expression in Breast Cancer Increases Sphingomyelin Levels

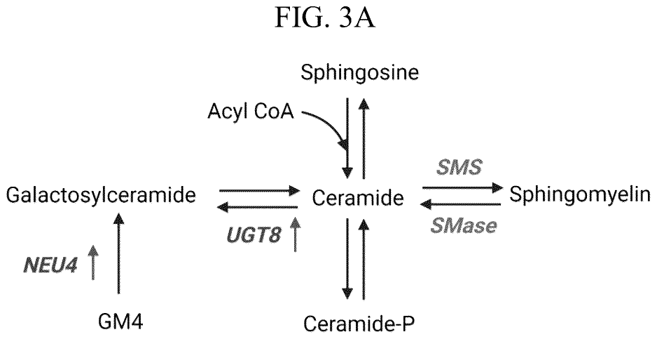

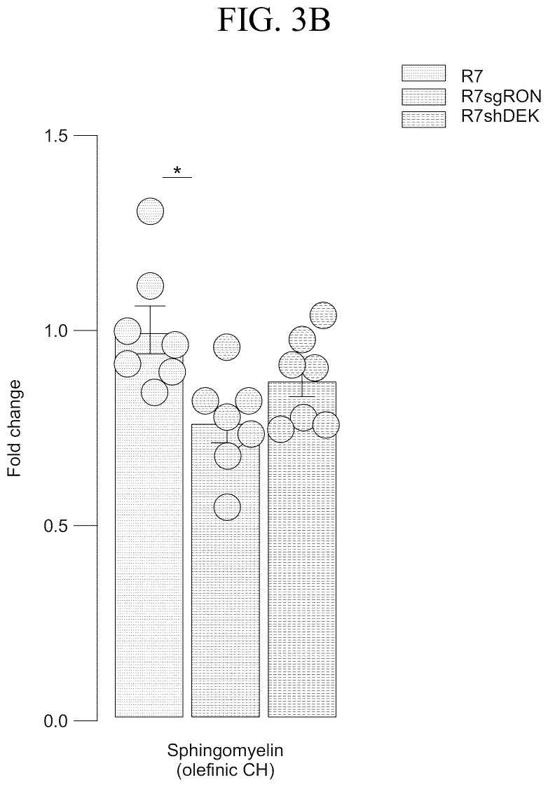

Sphingolipids are important structural constituents of cell membranes. Ceramide is a precursor and essential intermediate in the synthesis and metabolism of all sphingolipids (FIG. 3A). Sphingomyelin is produced by the transfer of phosphorylcholine from phosphatidylcholine to ceramide and constitutes the most abundant sphingolipid in mammalian cells. Sphingomyelin can also be hydrolyzed to produce ceramide, a pro-apoptotic molecule (FIG. 3A). Quantification of the 1H-NMR peaks at 5.70 ppm corresponding to the olefinic CH of the sphingomyelin showed that the R7sgRON cells had decreased sphingomyelin levels compared to both the R7 and R7shDEK cell lines (FIG. 3B). This implies that RON, but not DEK, potentially promotes sphingomyelin synthesis and breast cancer progression. Both ceramide and sphingomyelin act as regulatory molecules that inversely control cellular proliferation and tumor progression. Increased production (or reduced degradation) of sphingomyelin indirectly promotes cell proliferation due to decreased ceramide levels. Moreover, aberrant accumulation of sphingomyelin in the plasma membrane impairs membrane fluidity and permeability. As a consequence, sphingomyelin not only decreases cell-cell communication in favor of uncontrolled proliferation and invasion, but also impairs anticancer drug influx, contributing to chemotherapeutic resistance.

In addition, several sphingolipid enzymes play a key role in the ceramide-sphingomyelin homeostasis and cancer progression. UDP glycosyltransferase 8 (UGT8) mediates the synthesis of galactosylceramide from ceramide using UDP-galactose as a donor (FIG. 3A). Higher UGT8 mRNA and protein levels were observed in breast cancer metastases to the lung compared to their respective primary tumors. The overexpression of UGT8 and accumulation of its product galactosylceramide are associated with increased aggressiveness and worse prognosis in breast cancer. Notably, UGT8 has already been identified as part of a 6-gene signature that correlates with a higher risk for developing lung metastases and has been validated in 3 independent cohorts of breast cancer patients.

Finally, sphingomyelin synthase (SMS) mediates the formation of sphingomyelin from ceramide. High SMS expression leads to the concomitant accumulation of sphingomyelin and reduction of ceramide. An imbalance in ceramide-sphingomyelin homeostasis stimulates cancer cell proliferation by reducing ceramide-related apoptosis and induces tumor invasiveness and migration by enhancing epithelial-to-mesenchymal transition (EMT).

### DEK Regulates Glycerophospholipids Levels and Free Glycerol Availability

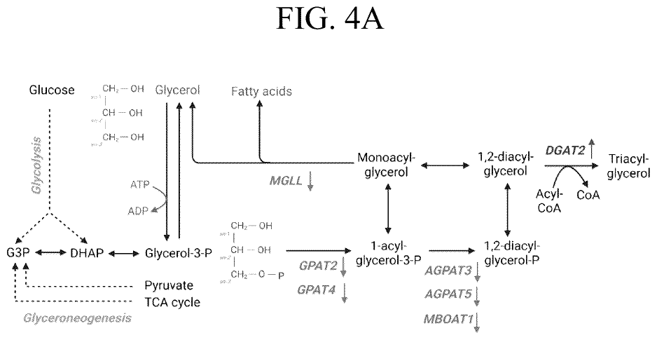

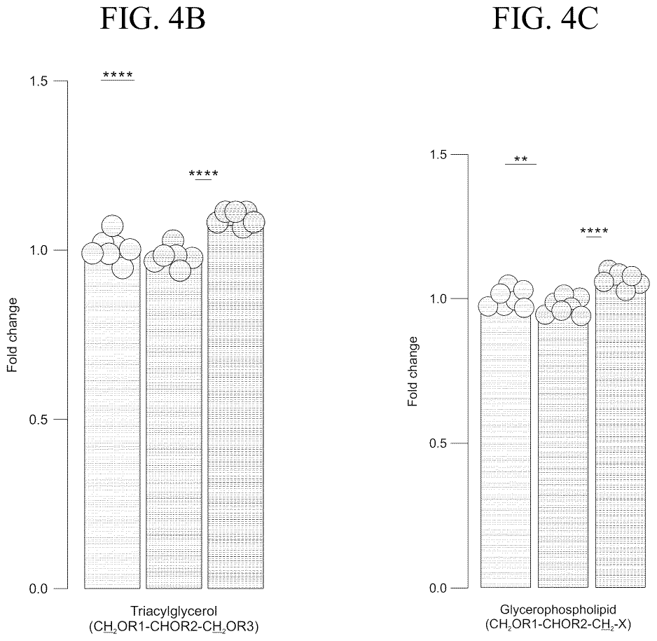

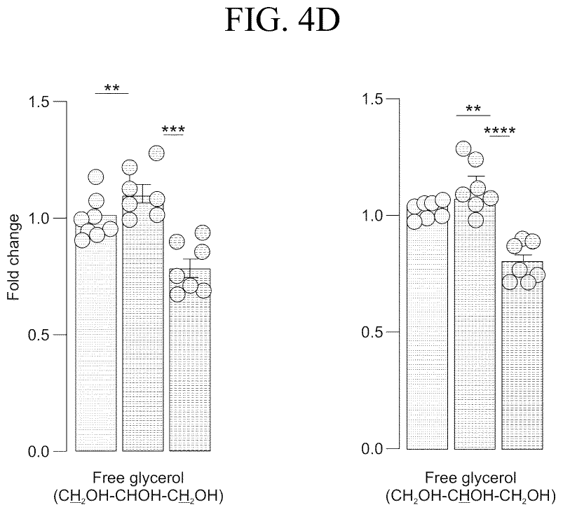

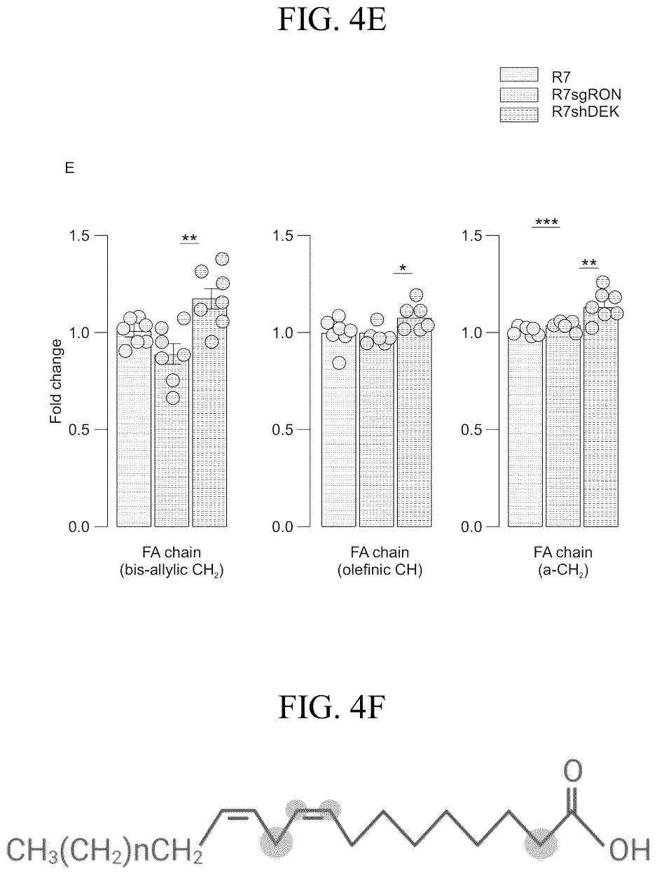

Intracellular glycerol is used as a building block for glycerophospholipids which are the major lipids in cell membranes and can be incorporated into vesicles for either intracellular or extracellular transport. The glycerol moiety of triacylglycerols (and other acylglycerols) can be generated from three sources: glucose, glycerol, or other metabolites such as pyruvate, alanine, lactate, or TCA cycle intermediates via glyceroneogenesis (FIG. 4A). Glycerol was identified, assigned, and quantified in several lipid classes. The 1H NMR peaks of the protons located at positions C1 and C3 of the glycerol backbone (CH2OR1-CHOR2-CH2OR3) of TAG generated two sets of peaks at 4.16 ppm and 4.45 ppm. The double doublet at 4.45 ppm was well resolved and isolated and thus integrated and used for quantification (FIG. 4B). In addition, the glyceryl C3H2 group (CH2OR1-CHOR2-CH2-X) of glycerophospholipids was readily observed and used for quantification (FIG. 4C). In both cases, R7shDEK cell lines harbored significantly increased levels compared to the R7 and R7sgRON cell lines. In contrast, when comparing the NMR peaks derived from free glycerol, both C1H3 and C3H3 and C2H2 resonances showed that the R7shDEK cells had decreased free glycerol levels compared to both the R7 and R7sgRON cell lines, and there was no difference between glycerol levels in R7 and R7sgRON cell lines (FIG. 4D). These results suggest that DEK but not RON expression could lead to decreased levels of TAG and other glycerophospholipids and increased free glycerol levels.

Triacylglycerols (TAGs) are the major energy reservoir in animals. They contain three esters of glycerol with fatty acids (FA). The main source of free glycerol in cells is lipolysis, which occurs via the breakdown of triacylglycerols into glycerol and free fatty acids (FFA). Glycerol can be also produced directly from glycerol-3-phosphate via glycolysis (46) (FIG. 4A). FA, specifically the fatty acyl chains as components of complex lipids, are essential for maintaining cell membrane architecture. In fact, de novo FA synthesis (lipogenesis) has been recognized as a hallmark of malignancy. In breast cancer FA synthesis was reported to be heightened via the upregulation of several lipogenic enzymes, such as FASN or ACLY.

The main substrate for the biosynthesis of FA is acetyl-CoA, which is first carboxylated by acetyl-CoA carboxylase to form malonyl-CoA. The condensation of seven molecules of malonyl-CoA and one molecule of acetyl-CoA ultimately generates palmitate, a 16-carbon saturated FA. Palmitoyl-CoA is the main product of lipogenesis and can later be elongated and desaturated to produce other FA species. In cancer cells, as a consequence of an increased de novo lipogenesis, saturated and monounsaturated FA are the dominant species of FA. This provides an advantage for cancer progression and metastasis, since these forms of FA are more stable, less susceptible to peroxidation and ultimately more resistant to cellular damage. Hence, the composition of these FAs is also decisive for the survival of tumor cells. The decreased level of unsaturation in membranes may affect their properties and those of integral membrane proteins.

Quantification of the olefinic (C═CH), as well as the bis-allylic (—CH2) group, which is specific for linoleic acid, revealed that R7shDEK cells harbor significantly increased levels of unsaturated FAs (FIG. 4E, F) compared to the R7 and R7sgRON cell lines. This suggests that DEK expression decreases unsaturated FAs, thus decreasing membrane fluidity. In addition, the analysis of the α-methylene FA group showed that total FA levels are increased in the R7shDEK compared to R7 and R7sgRON. GPATs (Glycerol-3-phosphate acyltransferases) catalyze the first step of the de novo biosynthesis of TAG and glycerophospholipids, while the AGPATs (acylglycerolphosphate acyltransferase) and MBOAT1 (membrane bound O-acyltransferase domain containing 1) catalyze the second step. These enzymes have a central role as modulators of lipid homeostasis and storage. Specifically, downregulation of GPAT2 has been associated with malignancy, proliferation, and survival in breast cancer cell lines. Inhibition of MGLL (monoacylglycerol lipase), which is an enzyme involved in the generation of fatty acids and glycerophospholipids from monoacylglycerol, was associated with decreased infiltration into the blood brain barrier in triple negative breast cancer in mouse models. This contrasts with our findings below that decreased MGLL mRNA levels are associated with worse prognosis in breast cancer. However, it is important to note that mRNA levels do not necessarily reflect protein levels and/or enzyme activity.

### a Gene Signature of Lipid Metabolic Enzymes Predicts Breast Cancer, Ovarian and Lung Cancer Outcome

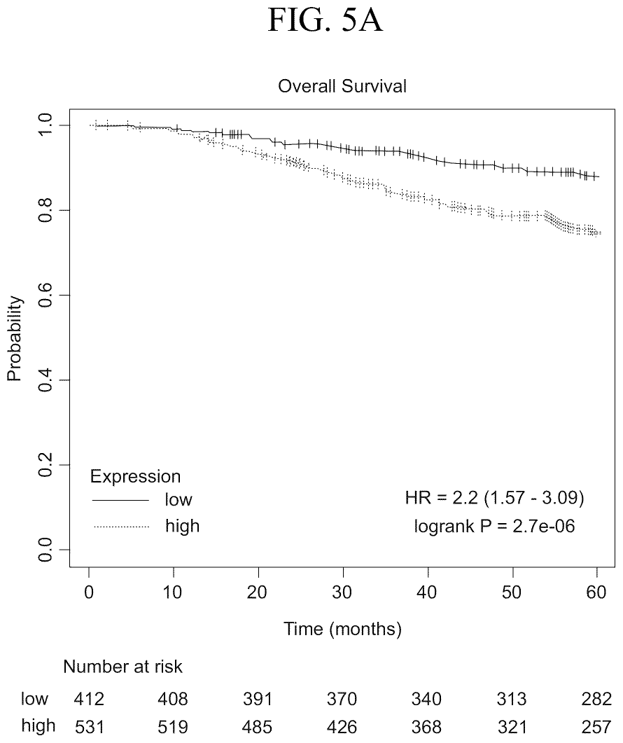

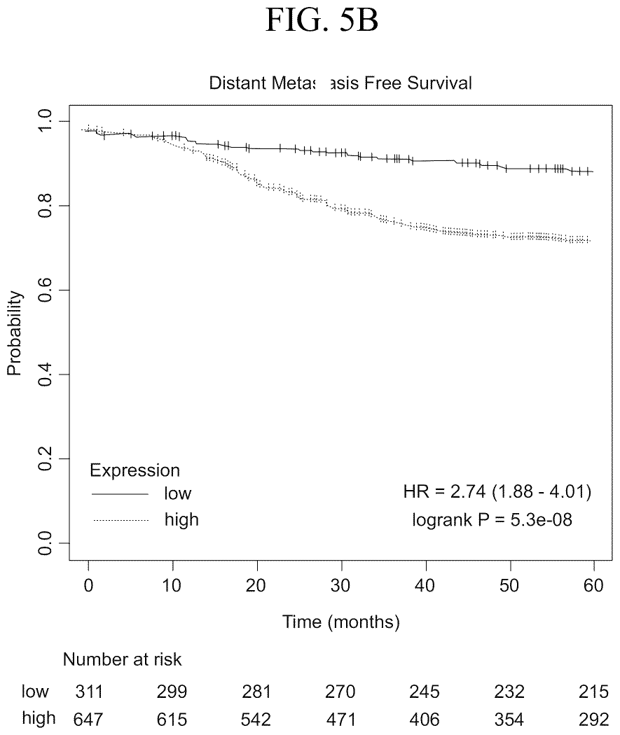

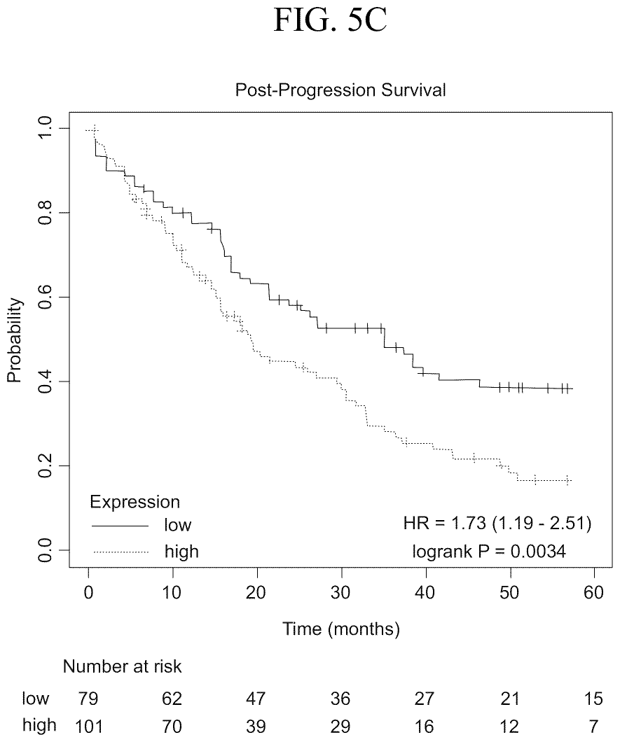

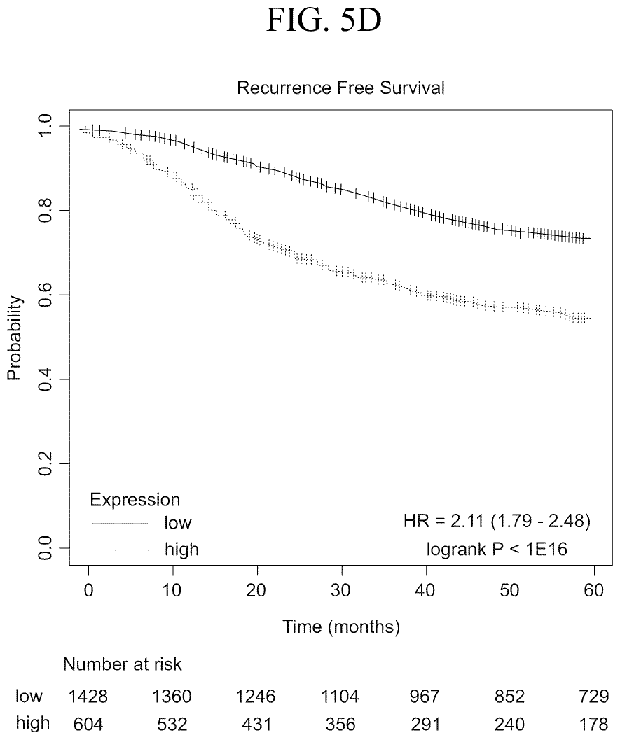

Genes involved in the metabolism of various lipid classes (fatty acids, cholesterol, steroids, sphingolipids, phospholipids, triglycerides, glycolipids, and cardiolipins) were examined. It was determined where the expression of genes encoding metabolic enzymes in these pathways was associated with worse outcomes in breast cancer outside of the RON/DEK context. We focused on Relapse Free Survival (RFS) since breast cancer outcomes worsen substantially upon relapse. Using the KMplot webtool, we investigated overall survival (OS, FIG. 5A), distant metastasis free survival (DMFS, FIG. 5B), post-progression survival (PPS, FIG. 5C), and recurrence-free survival (RFS, FIG. 5D) from the Gene Expression Omnibus (GEO)-derived dataset and identified a gene signature of 47 genes associated with significantly decreased patient survival regardless of breast cancer subtype. Table 1 shows the panel of genes, the respective lipid group associated with each gene, and up- or downregulation in patients with worse outcomes. Genes that are predictive of poor patient outcomes when upregulated include: enzymes involved in the hydrolyzation of acyl-CoA molecules, such as ACOT7 and ACOT13; DHCR7, an enzyme involved in the final steps of cholesterol biosynthesis; and ARSK, an enzyme involved in the modification of steroids, glycolipids, and carbohydrates. Genes that are predictive of good patient outcomes when upregulated include: ELOVL5, an enzyme involved in the elongation of fatty acids; BDH2, an enzyme involved in fatty acid beta oxidation; ETNK1, an enzyme required for the first step of phosphatidylethanolamine synthesis; and MGLL, an enzyme involved in the production of fatty acids and glycerol from acylglycerides.

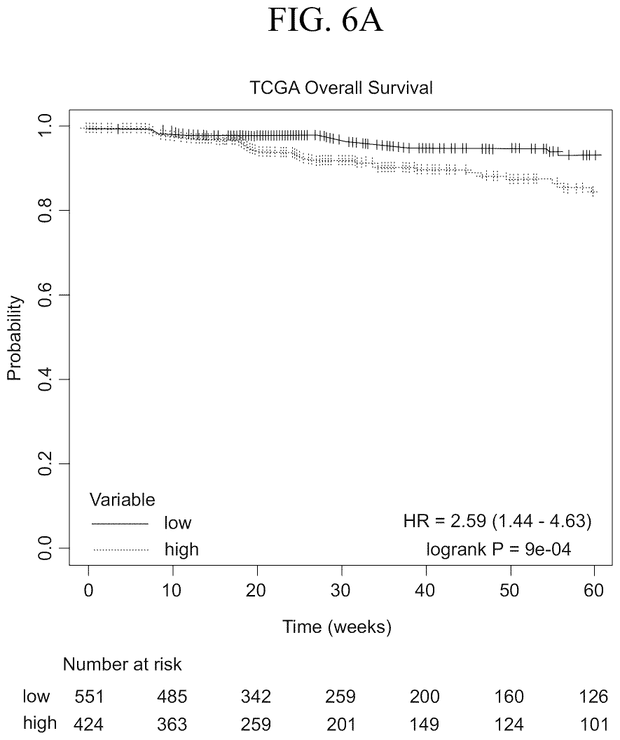

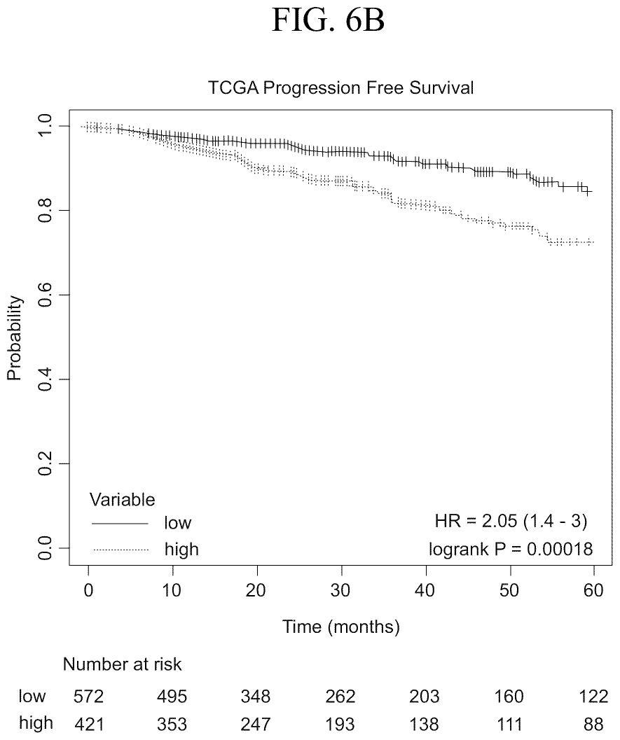

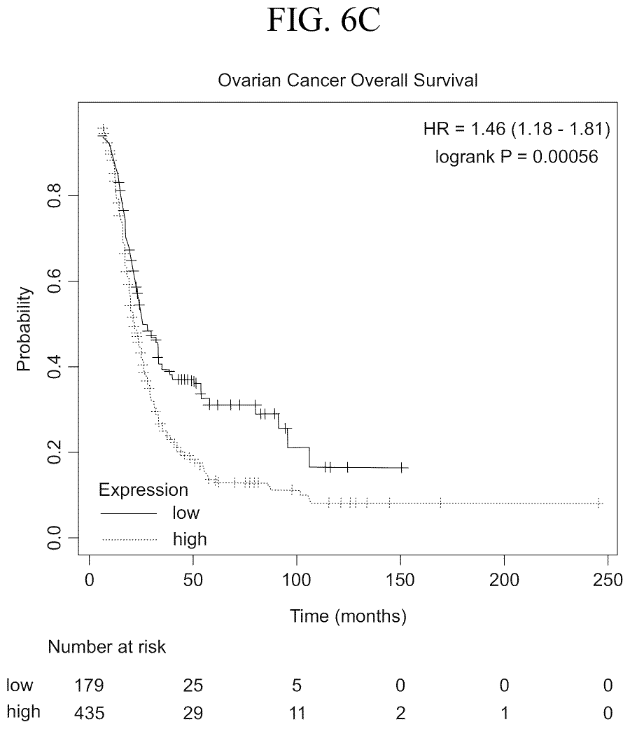

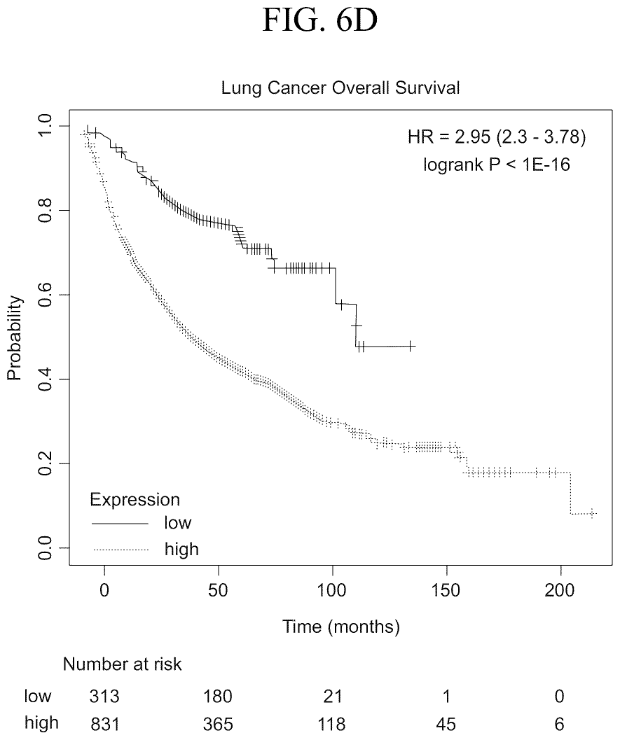

We next queried the gene signature using an independent Breast Cancer dataset derived from the TCGA Pancancer Atlas using overall survival (OS, FIG. 6A) and progression free survival (PFS, FIG. 6B), as well as Ovarian Cancer (FIG. 6C) and Lung Cancer datasets (FIG. 6D). This analysis recapitulated the results obtained from the dataset used in the KMplot webtool.

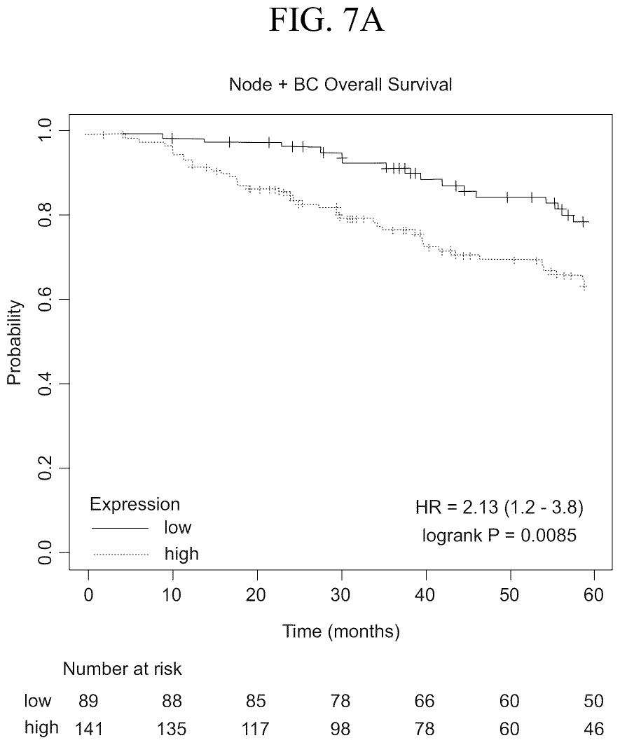

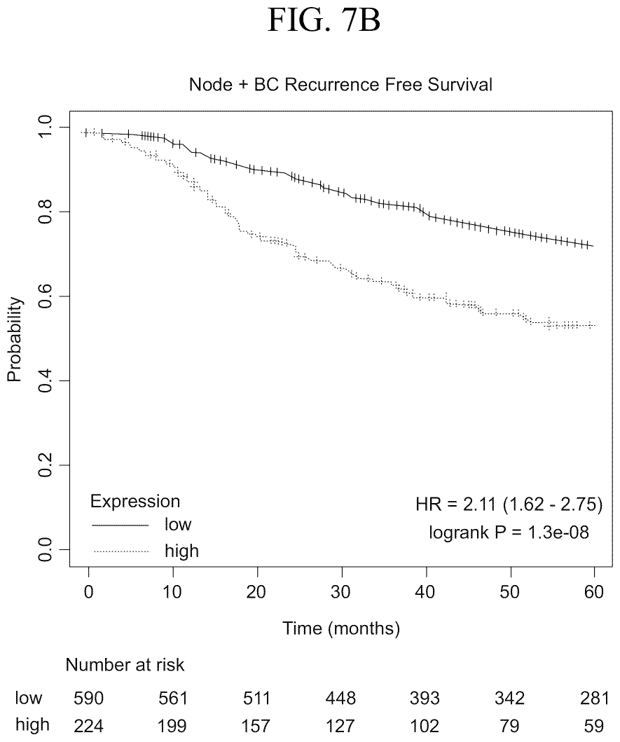

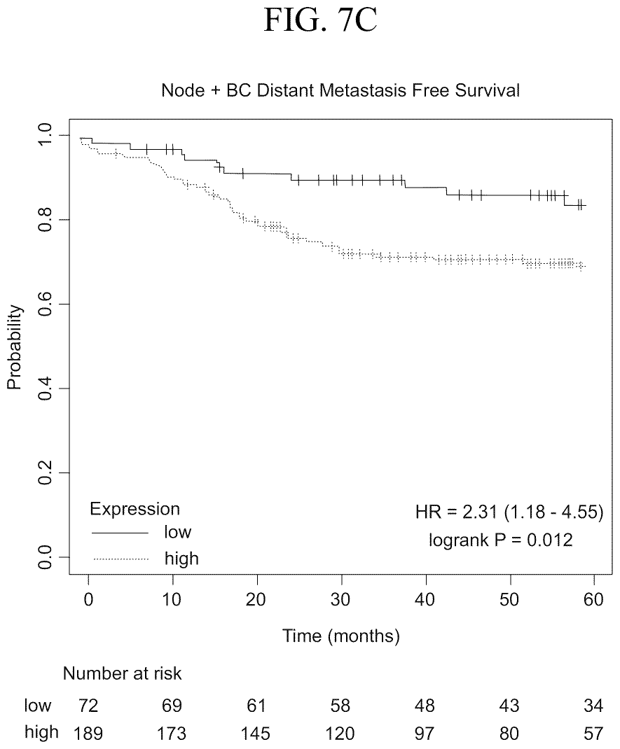

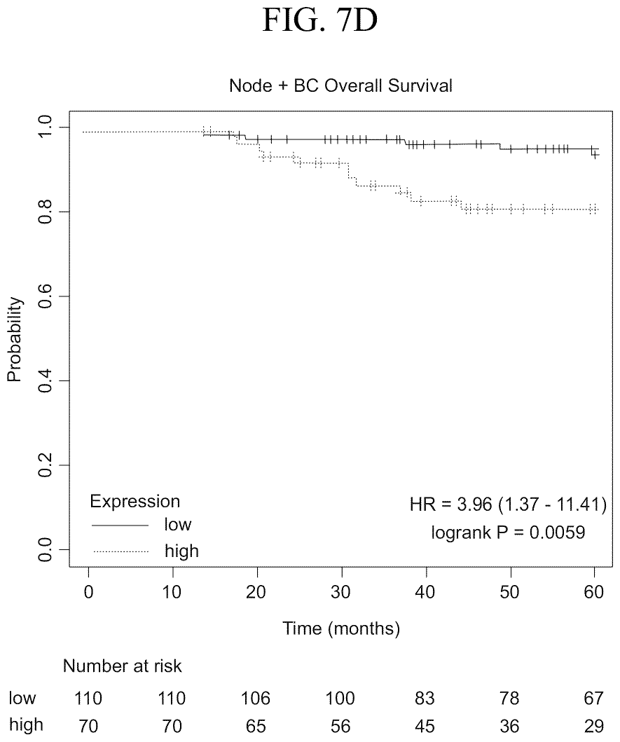

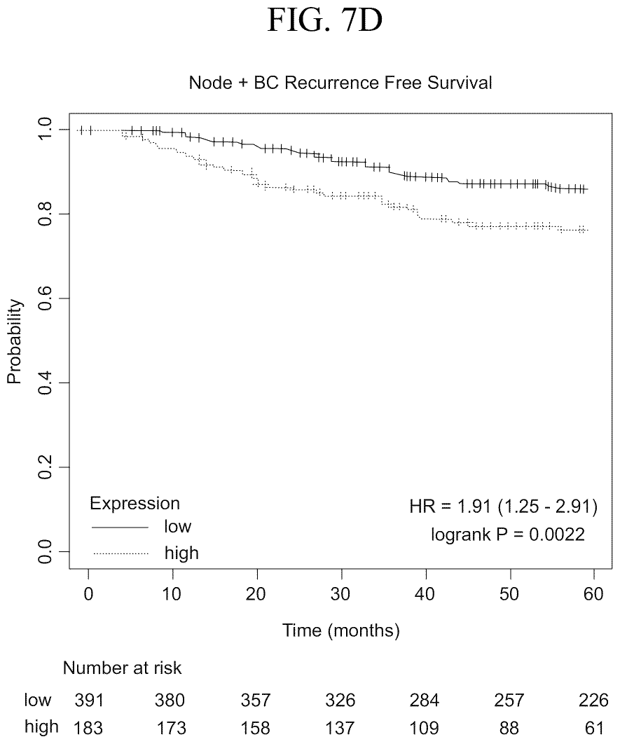

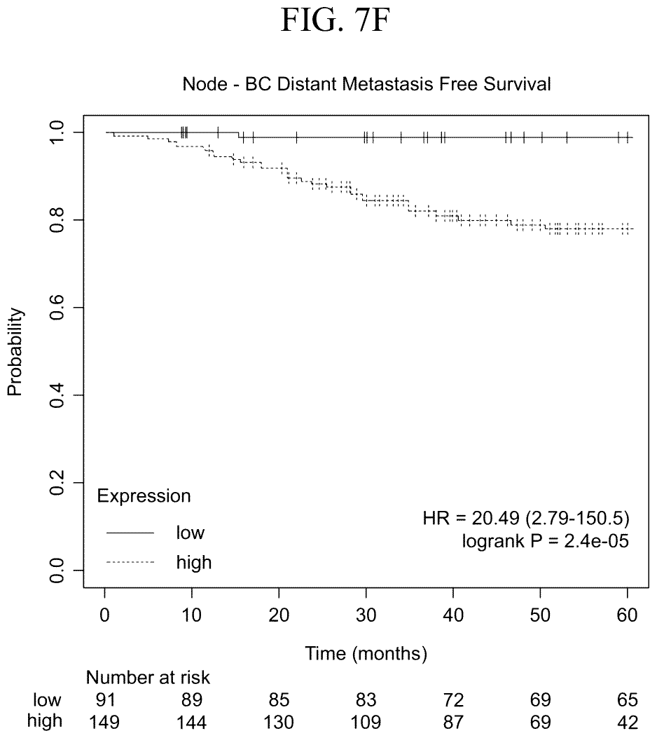

We further characterized the lipid gene signature in breast cancer by narrowing our focus to either Node + or Node − patients using data from KMplot.com (FIG. 7). The gene signature predicted worse overall survival (FIG. 7A, D), recurrence free survival (FIG. 7B, E), and distant metastasis free survival (FIG. 7C, F) regardless of nodal status, with a hazard ratio of 20.49 for distant free metastasis in Node—patients (FIG. 7F). Importantly, this implies that in patients with early stage (Node -) breast cancer, this gene signature is highly predictive of progression to metastatic disease.

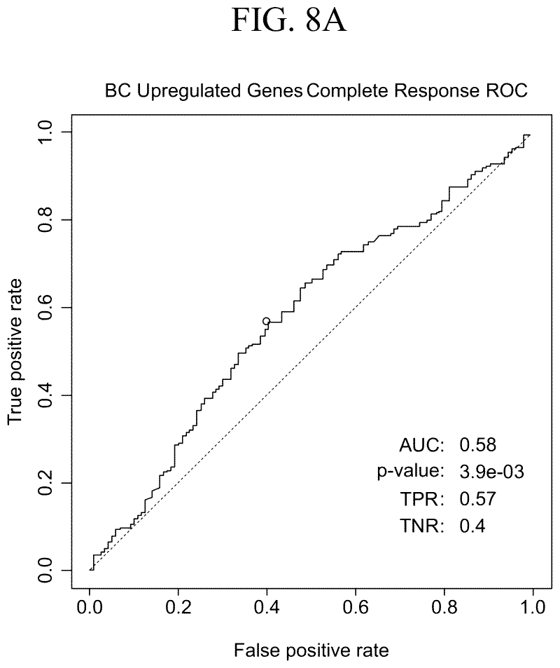

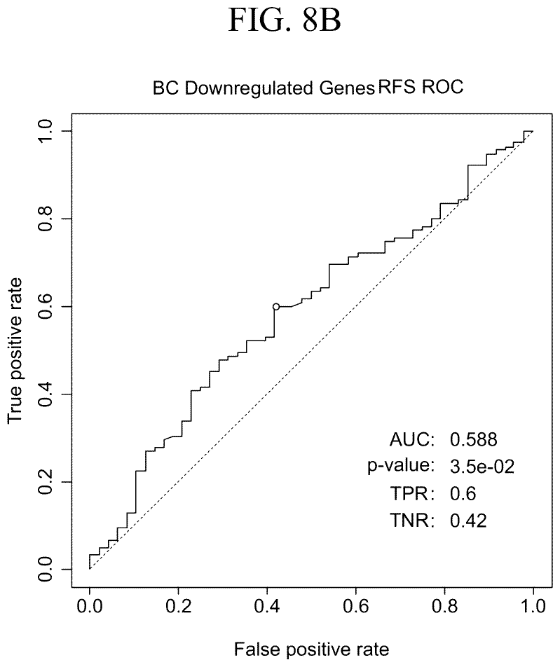

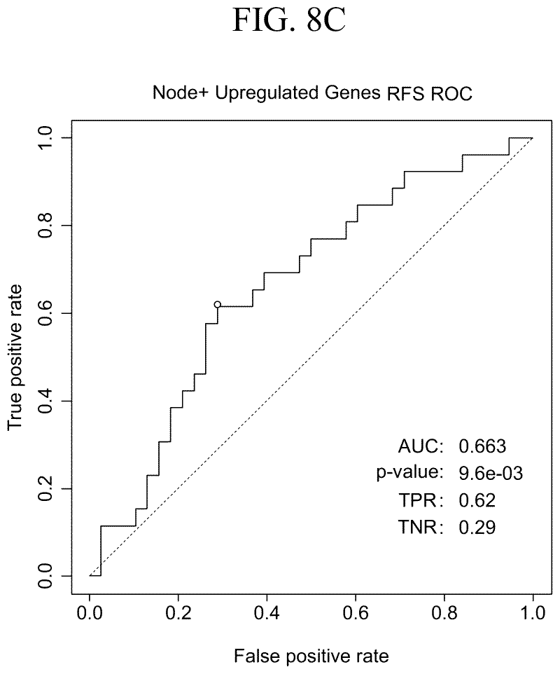

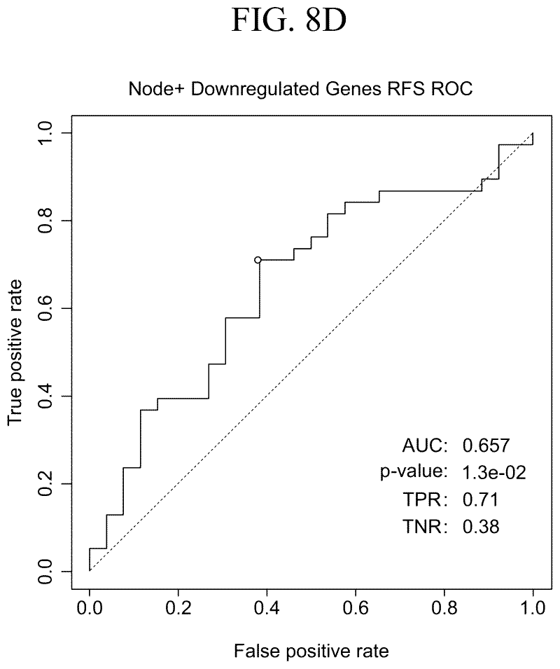

We then tested the predictive potential of the above lipid metabolism signature for breast cancer, ovarian cancer, colorectal cancer, and glioblastoma response to therapy using the ROCplot webtool. The genes were separated into two sets. One set included genes that were over-expressed in cancers that had recurred, while the other set included genes that were under-expressed in cancers that had recurred. Genes that were over-expressed were predictive of a complete response to chemotherapy in breast cancer (FIG. 8A), while genes that were under-expressed were predictive of relapse-free survival in breast cancer (FIG. 8B). Both sets of genes were predictive of relapse-free survival in node + breast cancer (FIG. 8C, D).

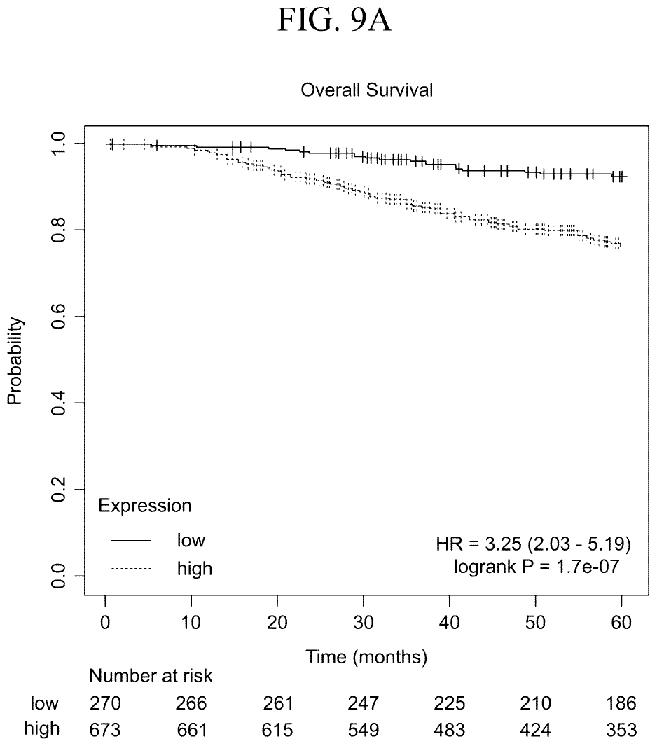

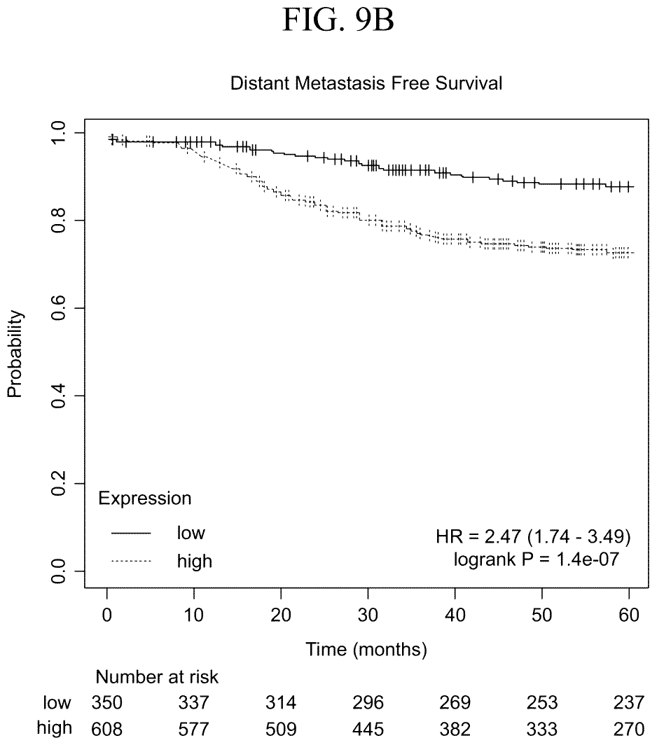

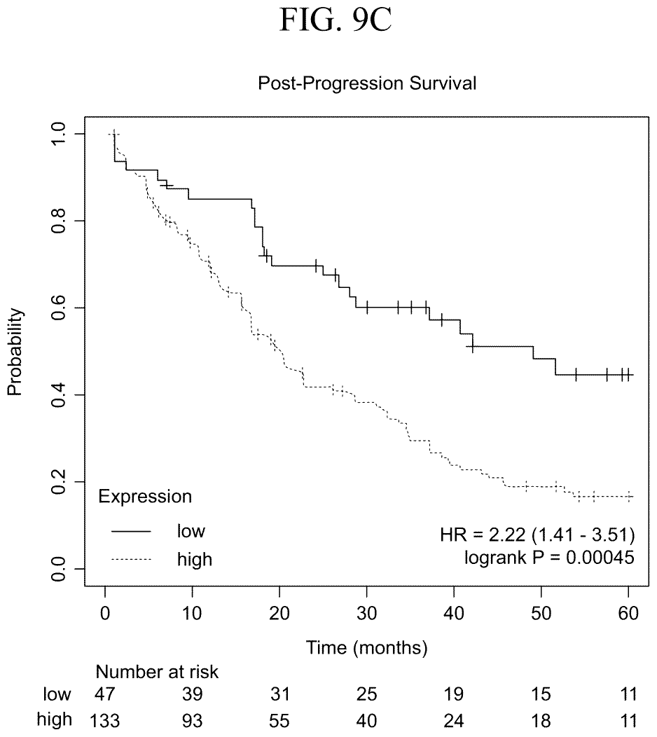

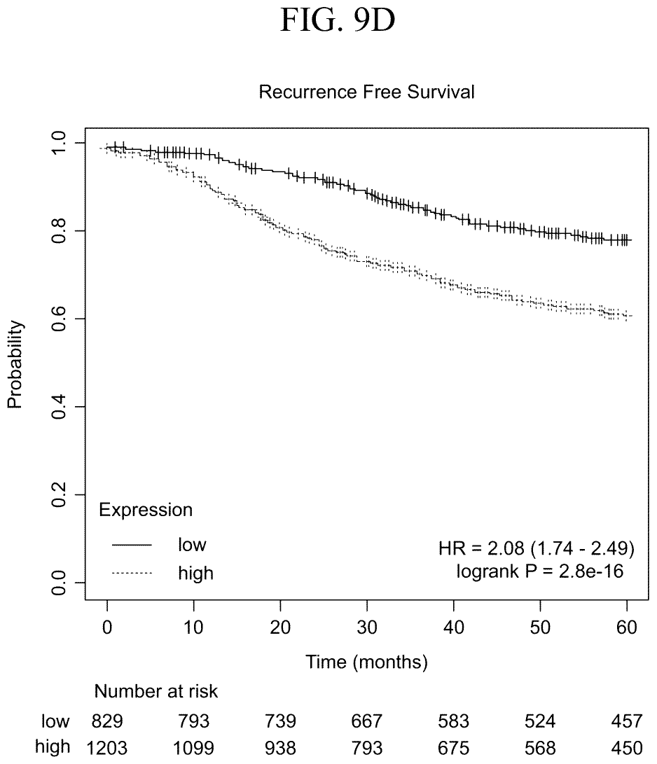

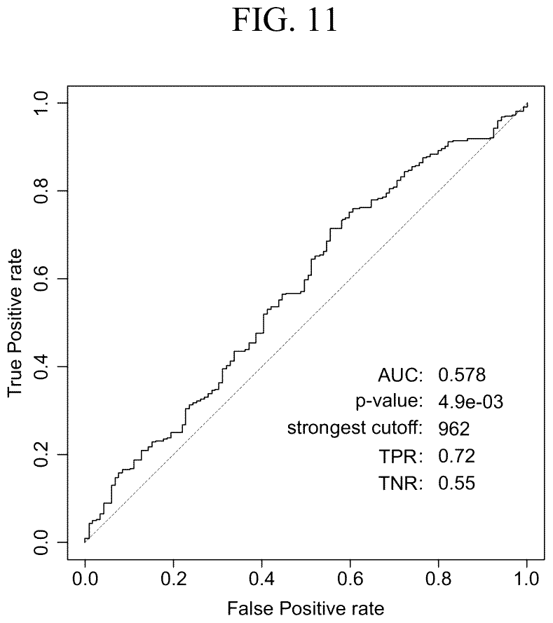

To further refine the lipid gene signature to its necessary components, we determined the minimal number of lipid metabolism related genes predictive of breast cancer outcomes, and arrived at four genes based on their RNAseq expression in R7 and T47D cell lines compared to their isogenic shRON counterparts: DHCR7, BDH2, ELOVL5, and ARSK. Compared to the above signature of 47 genes, these four genes were equally sufficient to predict overall survival, distant metastasis free survival, post-progression survival and recurrence free survival at 60 months (FIG. 9A-D). In addition, ROC analysis was performed using these four genes on the ROCplot webtool showing a prediction of complete response to chemotherapy similar to that of the 47 gene signature (FIG. 11).

In certain embodiments of the present invention, the method further comprises administering to the subject a cancer treatment. The cancer treatment can be chemotherapy, biological therapy, radiotherapy, immunotherapy, hormone therapy, anti-vascular therapy, cryotherapy, toxin therapy and/or surgery, including combinations thereof. In one embodiment, the chemotherapy can be taxol or sorafenib.

### EXAMPLES

### Materials and Methods

### Example 1—Cell Culture

The well characterized and widely published R7 (control), R7sgRON (RON targeted), R7shDEK (DEK targeted) murine breast cancer cell lines were cultured as previously described in complete Dulbecco's Modified Eagle Medium (DMEM) containing 5% FBS, 1% penicillin-streptomycin, and 0.2% fungizone. R7sgRON cells were obtained from serial dilution of px458-sgRON transfected R7 cells. Px458 was obtained from Addgene (48138), while RON sgRNA was obtained from IDT (sgRON sequence: ACCTGCAGCTCACCCTTCTAC). R7shDEK cells were maintained with 1 g/mL of puromycin for selection. For NMR experiments, cells were seeded in 10 cm-plates in complete DMEM containing 5% dialyzed FBS.

### Example 2—Cell Collection and Processing

The three isogenic breast cancer cell lines were plated and incubated for 24 h. At the time of collection, cells were 80-90% confluent; medium was aspirated, and the cells were washed and quenched as per prior protocols. Polar and nonpolar metabolites were extracted using the solvent partition method with acetonitrile:water:chloroform (CH3CN:H2O:CHCl3) at ratios 2: 1.5: 1 (V/V). A mixture of chloroform and methanol (1:1) containing 1 mM butylated hydroxytoluene (BHT) was added to the lipid fraction for storage at −80° C. The upper aqueous phases (polar metabolites) were lyophilized (CentriVap Labconco) and the lower organic phases (lipidic nonpolar metabolites) were dried in a SpeedVac at room temperature.

### Example 3—NMR Spectroscopy

Dried organic phases (lipids) were reconstituted in 220 μL of 100% methanol-d4 containing 0.05% v/v of tetramethylsilane (TMS) (Cambridge Isotopes Lab. Andover, MA), vortexed and centrifuged at room temperature. 200 μL of the supernatant were transferred into 3 mm NMR tube. All NMR spectra were recorded at 288 K on a Bruker Avance III HD 600 MHz spectrometer (Bruker Biospin) equipped with a 5 mm Broad Band Observed (BBO) Prodigy probe. For each sample, one-dimensional (1D) 1H-NMR experiments were acquired using the noesygpprld pulse sequence with presaturation of the residual water resonance using a 25 Hz bandwidth, 512 transients, a 15-ppm spectral width, a 4.0 s relaxation delay, and a 2.0 s acquisition time resulting in 44640 data points. Prior to Fourier transformation, each 1H spectrum was zero-filled to 128 K data points and apodized with a 1 Hz exponential line-broadening function. All spectra were recorded and transformed with the use of Topspin 3.6.2 software (Bruker BioSpin, USA) and processed (phased and baseline corrected) using MestReNova software (MNova v12.0.3, Spain). Spectra were internally calibrated to the methyl group of the TMS at 0 ppm. Representative lipid families (glycerophospholipids, sterols, sphingolipids, glycerophospholipids, and fatty acids) were identified and assigned by using in-house databases, pure standards, and literature reports. Additionally, for selecting samples, 2D 1H-1H TOtal Correlation SpectroscopY (TOCSY) experiments were recorded to facilitate and confirm the identification of analytes. The area of each assigned lipid class was manually integrated using global spectra deconvolution (GSD) algorithm available in MestReNova software (MNova v12.0.3, Spain), as previously described.

### Example 4—Statistical Analysis

To assess the relative abundance of each species of lipids identified compared to the R7 (control) group, each peak area was internally normalized to the area of the (CH3)3-N+ choline resonance at 3.22 ppm in the same spectrum. Principal component analysis (PCA) was carried out on the data scaled to unit variance by dividing each variable by its standard deviation (SD) using the R software. Univariate statistical analysis was used to determine the relevant spectral regions responsible for the discrimination between the groups. One-way ANOVA was used to generate pairwise comparisons between the R7, R7sgRON and R7shDEK groups. Benjamine-Hochberg procedure was used to control the false discovery rate of the pairwise comparison at q=0.05. The control group was set to 1 for comparison and the data were expressed as fold change of the relative amount of the different lipid adducts. Data are displayed as mean±standard error of the mean (SEM).

### Example 5—Design and Validation of a Predictive Gene Signature

Select genes encoding enzymes involved in lipid metabolism (fatty acids, cholesterol, steroids, sphingolipids, phospholipids, triglycerides, glycolipids, and cardiolipins) were utilized to stratify breast cancer patient outcomes with respect to relapse-free survival (RFS) in the Gene Expression Omnibus-derived KMplot datasets. A comprehensive list of genes associated with these lipids-related pathways was obtained. This initial list was then refined to generate a gene signature based on the log-rank p-value for RFS, using the KMplot webtool. Stratification into low and high gene expression groups was performed using a sliding cutoff approach from the KMplot webtool which optimizes Hazard Ratio (HR) values. Genes with worse outcomes from lower expression had their values inverted for the gene signature. Genes whose expression statistically significantly stratified RFS (p value<0.05) were used to construct the lipid gene signature through the arithmetic mean of each gene. Each signature was subjected to sliding cutoff. Additionally, the gene signature was used to test breast cancer patient overall survival (OS), distant metastasis free survival (DMFS), and post-progression survival (PPS) in the Gene Expression Omnibus-derived KMplot datasets. The signature was then validated using the Cancer Genome Atlas (TCGA) Pan Cancer dataset Overall Survival (OS) and Progression Free Survival (PFS). Finally, we tested the gene signatures for their capacity to predict response to chemotherapy in node-positive breast cancer patients using receiver-operator characteristic (ROC) analysis from the ROCplot webtool analyzing GEO-derived breast cancer patient data. For the ROC analysis, genes that were upregulated and led to worse outcomes were tested separately from genes that were downregulated. Finally, the gene signature was tested using the KMplot dataset for ovarian and lung cancer and the ROCplot webtool for 6 month survival in ovarian cancer.

### Tables

TABLE 1
Lipid Metabolism Genes with Prognostic Capacity. Genes are
displayed with a notation of upregulated (Up) or downregulated
(Down) in breast cancer in association with worse
outcomes. The Log-rank p-value for Kaplan-Meier
survival curves obtained from the Kmplot webtool (23) are
included for Relapse Free Survival (RFS), Overall Survival (OS), Distant
Metastasis Free Survival (DMFS), and Post-Progression Survival (PPS).

| | | Upre- | | | | |
|---|---|---|---|---|---|---|
| | | gulated | | | | |
| | | (Up) or | | | | |
| | | down- | | | | |
| | | regu- | | | | |
| | | lated | Log-rank p-value | | | |
| Lipid Class | Gene | (Down) | RFS | OS | DMFS | PPS |
| Fatty Acids | ACOT7 | Up | 9e−05 | 0.055 | 0.031 | 0.0014 |
| | ACOT13 | Up | 1.7e−08 | 0.071 | 0.00024 | 0.045 |
| | ME1 | Up | 2.9e−15 | 0.001 | 4.3e−08 | 0.02 |
| | ANGPTL4 | Up | 5.7e−11 | 1.8e−06 | 4.9e−07 | 0.083 |
| | OLAH | Down | 0.018 | 0.12 | 0.31 | 0.09 |
| | ACACB | Down | <1e−16 | 0.0022 | 6e−10 | 0.12 |
| | IVD | Down | 3.1e−16 | 0.00043 | 0.00057 | 0.037 |
| | FASN | Down | 3.5e−06 | 0.018 | 5.7e−05 | 0.022 |
| | ACSL6 | Down | 7.2e−06 | 0.00033 | 0.0027 | 0.12 |
| | ELOVL5 | Down | <1e−16 | 6.3e−07 | <1e−16 | 0.00013 |
| | CPT1C | Down | 3.1e−05 | 0.092 | 0.043 | 0.031 |
| | CPT1B | Down | 1.4e−15 | 0.012 | 0.14 | 0.016 |
| | ACADSB | Down | <1e−16 | 1.8e−10 | 1.3e−09 | 0.023 |
| | ACAA2 | Down | 2.8e−05 | 0.3 | 0.037 | 0.038 |
| | ACOX1 | Down | 0.0074 | 0.094 | 0.13 | 0.14 |
| | PTGR1 | Down | 1.9e−05 | 0.0024 | 0.007 | 0.0019 |
| | FAAH | Down | <1e−16 | 0.0089 | 2.1e−05 | 0.18 |
| | ACSF2 | Down | <1e−16 | 2.8e−05 | 9.1e−11 | 0.00033 |
| | BDH2 | Down | 6.9e−11 | 0.0018 | 6.5e−05 | 0.042 |
| | ACOT11 | Down | 9e−05 | 0.055 | 0.031 | 0.0014 |
| | MBOAT1 | Down | 2.3e−15 | 5e−07 | 1.5e−05 | 0.085 |
| Cholesterol | DHCR7 | Up | 3.3e−15 | 8.5e−09 | 9.7e−10 | 0.0017 |
| | MVK | Down | 3e−14 | 0.062 | 0.0011 | 0.11 |
| | STARD3 | Down | 6.8e−06 | 1e−04 | 0.0023 | 0.00088 |
| | OSBPL5 | Down | 9.4e−12 | 0.01 | 0.062 | 0.36 |
| Steroid | SRD5A1 | Up | 9.5e−14 | 0.0015 | 2.4e−10 | 0.0019 |
| | HSD17B2 | Up | 5.3e−05 | 2.7e−05 | 3.9e−05 | 0.0039 |
| | NCOA1 | Down | 3e−10 | 0.00078 | 1.3e−06 | 0.16 |
| Sphingolipids | UGT8 | Up | 3.4e−05 | 0.0061 | 0.021 | 0.019 |
| Phospholipids | LCLAT1 | Up | 1.8e−05 | 0.079 | 0.035 | 0.26 |
| | PLAAT1 | Up | <1e−16 | 3.8e−05 | 2.2e−12 | 0.0057 |
| | AGPAT3 | Down | 7e−12 | 0.0046 | 0.0017 | 0.29 |
| | AGPAT5 | Down | 1.1e−06 | 0.0012 | 0.051 | 0.028 |
| | GPAT4 | Down | 2.8e−12 | 0.24 | 0.28 | 0.31 |
| | INPP4B | Down | 3e−12 | 0.0034 | 8.8e−10 | 0.025 |
| | ETNK1 | Down | 1.4e−05 | 0.00041 | 0.022 | 0.36 |
| | PLAAT5 | Down | 1.1e−10 | 0.0064 | 0.00037 | 0.068 |
| | PITPNM2 | Down | 6e−09 | 0.0011 | 0.052 | 0.0028 |
| | SLC44A4 | Down | <1e−16 | 0.00052 | 1.2e−09 | 0.14 |
| Triglycerides | DGAT2 | Up | 0.0021 | 0.00033 | 0.0071 | 0.016 |
| | APOA5 | Down | 7.8e−08 | 0.026 | 0.074 | 0.021 |
| | GPAT2 | Down | 0.0025 | 0.34 | 0.023 | 0.037 |
| | MGLL | Down | 0.003 | 0.0022 | 0.21 | 0.24 |
| Glycolipids | ARSK | Up | 5.7e−14 | 0.036 | 0.032 | 0.11 |
| | NEU4 | Up | 4.4e−06 | 0.014 | 0.041 | 0.16 |
| Cardiolipins | PLD6 | Down | 9.9e−08 | 0.13 | 0.019 | 0.014 |

TABLE 2

| | Upregulated (Up) or |
|---|---|
| Gene | downregulated (Down) |
| ELOVL5 | Down |
| BDH2 | Down |
| DHCR7 | Up |
| ARSK | Up |

TABLE 3

| | | | | P Value (8 | | BH | |
|---|---|---|---|---|---|---|---|
| | | | | significant | | critical | |
| Lipids | Comparison | Summary | P Value | digits) | Rank | value | New P Value |
| ¹H_Triacylglycerol (CH₂OR1- | RON vs. DEK | **** | <0.0001 | 0.00000076 | 1 | 0.00167 | 0.0000228 |
| CHOR2-CH₂OR3) | | | | | | | |
| ¹H_Free Glycerol (CH₂OH-CHOH- | RON vs. DEK | **** | <0.0001 | 0.00000495 | 2 | 0.00333 | 0.00007425 |
| CH₂OH) | | | | | | | |
| ¹H_Cholesterol, C26, C27-CH₃ | RON vs. DEK | **** | <0.0001 | 0.00000513 | 3 | 0.00500 | 0.0000513 |
| ¹H_Triacylglycerol (CH₂OR1- | R7 vs. DEK | **** | <0.0001 | 0.00001918 | 4 | 0.00667 | 0.00014385 |
| CHOR2-CH₂OR3) | | | | | | | |
| ¹H_Glycerophospholipid | RON vs. DEK | **** | <0.0001 | 0.00004388 | 5 | 0.00833 | 0.00026328 |
| (CH₂OR1-CHOR2-CH₂-X) | | | | | | | |
| ¹H_Free Glycerol (CH₂OH-CHOH- | RON vs. DEK | **** | <0.0001 | 0.00005343 | 6 | 0.01000 | 0.00026715 |
| CH₂OH) | | | | | | | |
| ¹H_Cholesterol, C18-CH₃ | RON vs. DEK | **** | <0.0001 | 0.00008113 | 7 | 0.01167 | 0.0003477 |
| ¹H_FA chain (α-CH₂) | R7 vs. DEK | *** | 0.0002 | 0.00023200 | 8 | 0.01333 | 0.00087 |
| ¹H_Free Glycerol (CH₂OH-CHOH- | R7 vs. DEK | *** | 0.0004 | 0.00035309 | 9 | 0.01500 | 0.001176967 |
| CH₂OH) | | | | | | | |
| ¹H_FA chain (bis-allylic CH₂) | RON vs. DEK | ** | 0.0015 | 0.00148275 | 10 | 0.01667 | 0.00444825 |
| ¹H_Cholesterol, C26, C27-CH₃ | R7 vs. RON | ** | 0.002 | 0.00195841 | 11 | 0.01833 | 0.005341118 |
| ¹H_Free Glycerol (CH₂OH-CHOH- | R7 vs. DEK | ** | 0.0022 | 0.00223924 | 12 | 0.02000 | 0.0055981 |
| CH₂OH) | | | | | | | |
| ¹H_Glycerophospholipid | R7 vs. DEK | ** | 0.0029 | 0.00287391 | 13 | 0.02167 | 0.0066321 |
| (CH₂OR1-CHOR2-CH₂-X) | | | | | | | |
| ¹H_Cholesterol, C18-CH₃ | R7 vs. RON | ** | 0.0033 | 0.00327706 | 14 | 0.02333 | 0.007022271 |
| ¹H_FA chain (α-CH₂) | RON vs. DEK | ** | 0.006 | 0.00595627 | 15 | 0.02500 | 0.01191254 |
| ¹H_Sphingomyelin (olefinic CH) | R7 vs. RON | * | 0.0149 | 0.01485461 | 16 | 0.02667 | 0.027852394 |
| ¹H_FA chain (olefinic CH) | RON vs. DEK | * | 0.016 | 0.01597639 | 17 | 0.02833 | 0.028193629 |
| ¹H_Cholesterol, C26, C27-CH₃ | R7 vs. DEK | * | 0.0271 | 0.02710859 | 18 | 0.03000 | 0.045180983 |
| ¹H_FA chain (bis-allylic CH₂) | R7 vs. DEK | ns | 0.0526 | 0.05256182 | 19 | 0.03167 | 0.082992347 |
| ¹H_FA chain (olefinic CH) | R7 vs. DEK | ns | 0.0725 | 0.07253425 | 20 | 0.03333 | 0.108801375 |
| ¹H_Free Glycerol (CH₂OH-CHOH- | R7 vs. RON | ns | 0.1209 | 0.12091666 | 21 | 0.03500 | 0.172738086 |
| CH₂OH) | | | | | | | |
| ¹H_Glycerophospholipid | R7 vs. RON | ns | 0.1539 | 0.15391348 | 22 | 0.03667 | 0.209882018 |
| (CH₂OR1-CHOR2-CH₂-X) | | | | | | | |
| ¹H_Free Glycerol (CH₂OH-CHOH- | R7 vs. RON | ns | 0.2181 | 0.21809961 | 23 | 0.03833 | 0.284477752 |
| CH₂OH) | | | | | | | |
| ¹H_Cholesterol, C18-CH₃ | R7 vs. DEK | ns | 0.2277 | 0.22771990 | 24 | 0.04000 | 0.284649875 |
| ¹H_Triacylglycerol (CH₂OR1- | R7 vs. RON | ns | 0.2301 | 0.23006336 | 25 | 0.04167 | 0.276076032 |
| CHOR2-CH₂OR3) | | | | | | | |
| ¹H_FA chain (bis-allylic CH₂) | R7 vs. RON | ns | 0.2421 | 0.24205187 | 26 | 0.04333 | 0.279290619 |
| ¹H_Sphingomyelin (olefinic CH) | R7 vs. DEK | ns | 0.2461 | 0.24610622 | 27 | 0.04500 | 0.273451356 |
| ¹H_FA chain (α-CH₂) | R7 vs. RON | ns | 0.3189 | 0.31887960 | 28 | 0.04667 | 0.341656714 |
| ¹H_Sphingomyelin (olefinic CH) | RON vs. DEK | ns | 0.3247 | 0.3246846 | 29 | 0.04833 | 0.335880621 |
| ¹H_FA chain (olefinic CH) | R7 vs. RON | ns | 0.7404 | 0.74038321 | 30 | 0.05000 | 0.74038321 |

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A method for predicting tumor recurrence in a subject diagnosed with cancer, said method comprising:
a. analyzing a tumor sample obtained from said subject by using an assay to determine the extent of expression of one or more genes selected from the group consisting of ACOT7, ACOT13, ME1, ANGPTL4, DHCR7, SRD5A1, HSD17B2, UGT8, LCLAT1, PLAAT1, DGAT2, ARSK and NEU4 in said tumor sample (“Group 1 genes”);
b. analyzing the tumor sample by using an assay to determine the extent of expression of one or more genes selected from the group consisting of OLAH, ACACB, IVD, FASN, ACSL6, ELOVL5, CPT1C, CPT1B, ACADSB, ACAA2, ACOX1, PTGR1, FAAH, ACSF, BDH2, ACOT11, MBOAT1, MVK, STARD3, OSBPL5, NCOA1, AGPAT3, AGPAT5, GPAT4, INPP4B, ETNK1, PLAAT5, PITPNM2, SLC44A4, APOA5, GPAT2, MGLL and PLD6 in said tumor sample (“Group 2 genes”); and
c. predicting an increased likelihood that said subject will exhibit tumor recurrence when one or more of the Group 1 genes are upregulated, one or more of the Group 2 genes are downregulated or a combination of Group 1 genes are upregulated and Group 2 genes are downregulated.

2. The method of claim 1 wherein a combination of at least two genes selected from the Group 1 genes, the Group 2 genes, or both Group 1 genes and Group 2 genes are utilized for predicting an increased likelihood of tumor recurrence.

3. The method of claim 1 wherein a combination of at least four genes selected from the Group 1 genes, the Group 2 genes, or both Group 1 genes and Group 2 genes are utilized for predicting an increased likelihood of tumor recurrence.

4. The method of claim 3 wherein the four genes are DHCR7, BDH2, ELOVL5, and ARSK.

5. The method of claim 1 wherein a combination of at least eight genes selected from the Group 1 genes, the Group 2 genes, or both Group 1 genes and Group 2 genes are utilized for predicting an increased likelihood of tumor recurrence.

6. The method of claim 1 wherein a combination of at least ten genes selected from the Group 1 genes, the Group 2 genes, or both Group 1 genes and Group 2 genes are utilized for predicting an increased likelihood of tumor recurrence.

7. The method of claim 1 wherein a combination of said genes are utilized for predicting tumor recurrence prior to or after cancer treatments.

8. The method of claim 1 wherein the cancer is selected from the group consisting of lung cancer, prostate cancer, testicular cancer, brain cancer, skin cancer, colon cancer, rectal cancer, gastric cancer, esophageal cancer, tracheal cancer, head and neck cancer, pancreatic cancer, liver cancer, breast cancer, ovarian cancer, lymphoid cancer, kidney cancer, cervical cancer, bone cancer, vulvar cancer and melanoma.

9. The method of claim 1 wherein the cancer is breast cancer.

10. The method of claim 1 wherein the cancer is node positive or node negative breast cancer.

11. The method of claim 1 wherein a combination of gene sets identified in Table 1 are used as a diagnostic for metastasis, tumor recurrence, or both, prior to or after cancer treatment.

12. The method of claim 1 wherein the tumor sample is a blood sample.

13. The method of claim 1 further comprising modifying the subject's cancer therapy regimen based on the prediction of an increased likelihood of tumor recurrence.
